SEQUENCE LISTING (5 sequences)

SEQ ID NO: 1 - protein, 458 aa, Homo sapiens
  MISC_FEATURE (152)..(152): Xaa at position 152 may be Glycine or Glutamate
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYXQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKXSDFASNCCSINSPPLYCDSEIDAELKNIL

SEQ ID NO: 2 - protein, 458 aa, Homo sapiens
  MISC_FEATURE (152)..(152): Xaa at position 152 may be Glycine or Glutamate
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYXQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPEATPTELAKLVNKXSDFASNCCSINSPPLYCDSEIDAELKNIL

SEQ ID NO: 3 - protein, 458 aa, Homo sapiens
  MISC_FEATURE (152)..(152): Xaa at position 152 may be Glycine or Glutamate
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYXQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPKELAKLVNKXSDFASNCCSINSPPLYCDSEIDAELKNIL

SEQ ID NO: 4 - protein, 83 aa, Artificial Sequence
  Synthetic Peptide
LLKKELSSFIDKGQELCADYSENTFTEYKKKLAERLKAKLPDATPTELAKLVNKXSDFSNCCSINSPPLYCDSEIDAELKNIL

SEQ ID NO: 5 - protein, 84 aa, Artificial Sequence
  Synthetic peptide
LLKKELSSFIDKGQELCADYSENTFTEYKKKLAERLKAKLPDATPKELAKLVNKXSDFASNCCSINSPPLYCDSEIDAELKNIL

(19) United States
(12) Patent Application Publication
(10) Pub. No.: US 2016/0120946 A1
(43) Pub. Date: May 5, 2016

(54) COMPOSITIONS COMPRISING GC-MACROPHAGE ACTIVATING FACTOR AND USES THEREOF

(71) Applicant: EFRANAT LTD., Rehovot (IL)

(72) Inventors: Ilana MARGALIT, Ramat Gan (IL); Michal SHAHAR, Rishon Lezion (IL); Sveta LIFSHITS, Mazkeret Batya (IL); Aya SPITZER, Ness-Ziona (IL)

(21) Appl. No.: 14/893,354

(22) PCT Filed: Jun. 9, 2014
(86) PCT No.: PCT/IL2014/050516

§ 371 Date: Nov. 23, 2015

Related U.S. Application Data
(60) Provisional application No. 61/832,867, filed on Jun. 9, 2013.

(51) Int. Cl. A61K 38/19

(52) U.S. Cl. CPC A61K 38/19

(57) ABSTRACT

A pharmaceutical composition that includes stable Gc macrophage activating factor (GcMAF) or a biologically active variant or fragment thereof and at least one pharmaceutically acceptable excipient selected from the group consisting of a surfactant and a synthetic water-soluble polymer having surface activity. These compositions are storage stable and are used for treating a disease or disorder associated with macrophage activation.

### FIELD OF THE INVENTION

The present invention relates to stable pharmaceutical compositions comprising Gc macrophage activating factor (GcMAF). The present invention relates in particular to storage-stable pharmaceutical compositions comprising GcMAF and at least one pharmaceutically acceptable surfactant and/or a synthetic water-soluble polymer having surface activity and uses thereof for treating diseases associated with macrophage activation.

### BACKGROUND OF THE INVENTION

Macrophage activation plays a crucial role in the development of inflammation and in the regulation of immune responses. Macrophage activation associated with inflammation requires participation of B and T lymphocytes and serum vitamin D-binding protein (also known as “group-specific component” or Gc protein).

Gc protein is a polymorphic glycoprotein of the α-2 macroglobulin fraction of human plasma having an apparent molecular weight of 52 kDa, normally constituting about 0.5% of the proteins in human plasma. Gc protein carries a trisaccharide composed of N-acetylgalactosamine with dibranched galactose and sialic acid termini. This oligosaccharide is hydrolyzed by the inducible membranous β-galactosidase of inflammation-primed B lymphocytes to yield a macrophage pro-activating factor. The macrophage pro-activating factor in turn is hydrolyzed by the membranous Neu-1 sialidase of T lymphocytes to yield the macrophage activating factor (GcMAF). Stepwise treatment of purified Gc protein with immobilized β-galactosidase and sialidase was also shown to generate GcMAF.

GcMAF has been shown to have a tumoricidal role. It was demonstrated that GcMAF activity in cancer patients was abolished or reduced because serum Gc protein in these patients was deglycosylated by serum α-N-acetylgalactosidase (Nagalase) secreted from cancerous cells. As a result, the deglycosylated Gc protein was not converted to GcMAF, and macrophage activation was reduced or abolished. Administering exogenous GcMAF to cancer patients having melanoma, prostate cancer, colorectal cancer, or metastatic breast cancer showed a curative effect of GcMAF by overcoming the shortage in active macrophages (Yamamoto et al., 2008, Int. J. Cancer, 122: 461-467; Yamamoto et al., Trans. Oncol. 2008, 1: 65-72). Similar effect on macrophage activation was observed in HIV-infected and osteoperotic patients treated with GcMAF.

U.S. Pat. No. 5,177,002 discloses process for producing a macrophage activating factor comprising contacting glycosylated human group-specific component in vitro with β-galactosidase, or β-galactosidase in combination with sialidase, α-mannosidase, or a mixture thereof, and obtaining the macrophage activating factor. U.S. Pat. No. 5,177,002 further discloses a method for inducing macrophage activation in an individual in need thereof comprising administering to the individual the human macrophage activating factor thus produced.

WO 93/07288 discloses a process for the production of a potent macrophage activating factor wherein animal vitamin D-binding protein is contacted in vitro (i) with β-galactosidase, or (ii) with β-galactosidase in combination with sialidase, α-mannosidase or a mixture thereof. WO 93/07288 further discloses methods for activating macrophages comprising administering to an animal the macrophage activating factor thus prepared.

WO 96/40903 discloses cloning of vitamin D-binding protein (Gc protein) and its small domain (also known as domain III) via baculovirus vector. The cloned Gc protein and the cloned domain III were treated with immobilized β-galactosidase and sialidase to yield macrophage activating factors, GcMAFc and CdMAF, respectively. WO 96/40903 further discloses uses of the macrophage activating factors for the treatment of cancer, human immunodeficiency virus (HIV) infection and osteoporosis.

WO 2012/137199 discloses pharmaceutical compositions comprising macrophage activating factor (MAF) essentially devoid of glycosidase enzymes and methods of use therefore for treating cancer or HIV-infected patients.

Pihl et al. (Basic & Clin Pharm & Toxicol., 2010, 107: 853-860) disclose pre-clinical toxicology experiments conducted on purified plasma-derived human Gc protein and indicate that Gc protein at a concentration of about 10 mg/ml in phosphate buffered saline (PBS) in the presence of 5% maltose was found to be stable for four years with full retention of the actin-binding capacity and with no significant change in the amount of Gc protein.

Pacini et al. (Cancer Immunol. Immunother., 2011, 60: 479-485) evaluated different GcMAF preparations for their potency to inhibit PGE₁-stimulated angiogenesis in chick embryo chorioallantoic membrane (CAM) assay and indicated that storage at 25° C. for 15 days decreased GcMAF potency by about 50%.

There is unmet need for storage-stable pharmaceutical compositions of GcMAF which maintain GcMAF chemical stability and biological activity.

### SUMMARY OF THE INVENTION

The present invention provides pharmaceutical compositions having improved stability upon storage comprising Gc protein derived macrophage activating factor (GcMAF) and at least one pharmaceutically acceptable surfactant and/or a synthetic water-soluble polymer having surface activity. The present invention further provides methods of treating a disease or disorder associated with macrophage activation comprising administering to a subject in need of such treatment said stable pharmaceutical compositions.

The present invention is based in part on the findings that storage of aqueous solutions of GcMAF at low concentrations results in significant loss of the chemical stability and biological activity of GcMAF when the solutions are stored at 4° C. or frozen and then thawed.

It is now disclosed that traditional pharmaceutically acceptable stabilizers of polypeptide, such as human serum albumin (HSA), arginine, and the sugar alcohol mannitol, were essentially ineffective, whether added alone or in combination, in stabilizing GcMAF in aqueous solutions of GcMAF (below 30 μg/ml).

The inventors of the present invention have unexpectedly discovered that addition of a pharmaceutically acceptable surfactant to an aqueous solution having a low concentration of GcMAF overcame the problem of loss of chemical stability and biological activity upon storage. For example, addition of a nonionic surfactant to an aqueous solution of 1 μg/ml GcMAF maintained GcMAF chemical stability during storage of up to three months at 4° C., 25° C., and even at 37° C. The inventors of the present invention have further disclosed that the nonionic surfactant was capable of maintaining GcMAF in solution after one or more cycles of freezing and thawing of a solution having a low concentration, e.g., 1 μg/ml, of GcMAF.

It is further disclosed that nonionic surfactants not only maintained GcMAF chemical stability during long-term storage of a solution of a low concentration of GcMAF, but also preserved GcMAF biological activity during such storage as measured by activation of macrophages.

The inventors of the present invention have further disclosed that ionic surfactants as well as water-soluble polymers having surface activity were effective in stabilizing GcMAF during long-term storage. However, polysaccharides such as hyaluronic acid or alginate, did not exert a significant stabilizing effect on GcMAF. Thus, the compositions of the present invention are highly advantageous as they maintain the chemical stability and biological activity of GcMAF after long-term storage, i.e., after at least six months at 4° C., when formulated as a liquid formulation of a low concentration (˜100 ng/ml to ˜1 mg/ml) of GcMAF. Also, the compositions of the present invention simplify the handling and delivering of GcMAF and enable freezing the compositions with essentially little or no effect on GcMAF chemical stability once thawed, thus further avoiding the need to use the compositions immediately after thawing.

According to a first aspect, the present invention provides a pharmaceutical composition comprising stable or stabilized Gc macrophage activating factor (GcMAF) or a biologically active variant or fragment thereof and at least one pharmaceutically acceptable excipient selected from the group consisting of a surfactant and a water-soluble polymer.

According to some embodiments, the stable GcMAF is selected from the group consisting of human GcMAF and animal GcMAF having independently an N-acetylgalactosamine group linked to an amino acid residue. According to one embodiment, the stable GcMAF is human GcMAF having an N-acetylgalactosamine group linked to an amino acid residue. According to additional embodiments, the stable GcMAF comprises the amino acid sequence as set forth in any one of SEQ ID NOs: 1 to 3. According to further embodiments, the stable GcMAF fragment comprises the amino acid sequence corresponding to amino acids 400-435 of the Gc protein. According to yet further embodiments, the stable GcMAF fragment consists of the amino acid sequence as set forth in SEQ ID NO:4 or SEQ ID NO:5.

According to additional embodiments, the stable GcMAF is present at a concentration ranging from about 100 ng/ml to about 1 mg/ml when the composition is in the form of a liquid, alternatively GcMAF is present at a concentration ranging from about 100 ng/ml to about 300 μg/ml, further alternatively from about 200 ng/ml to about 30 μg/ml, and still further alternatively from about 200 ng/ml to about 1 μg/ml when the composition is in the form of a liquid. Each possibility is a separate embodiment of the invention. According to a certain embodiment, GcMAF is present at a concentration ranging from about 200 ng/ml to about 1 μg/ml when the composition is in the form of a liquid.

According to further embodiments, the surfactant is selected from the group consisting of nonionic surfactants, anionic surfactants, cationic surfactants, amphoteric surfactants and zwitterionic surfactants.

According to some embodiments, the nonionic surfactant is selected from the group consisting of sorbitan fatty acid esters, polyoxysorbitan fatty acid esters, polyoxyalkylene higher alcohol ethers, and polyoxyalkylene higher alcohol esters. Examples of nonionic surfactants include, but are not limited to, polyoxyethylene sorbitol esters, polyoxyethylene isooctylphenyl ethers, polyoxyethylene nonylphenyl ethers, polyoxyethylene dodecyl ethers, octyl glucoside, and alkyl maltoside. According to yet further embodiments, the nonionic surfactant is selected from the group consisting of polysorbate 80 (TWEEN® 80), polysorbate 60 (TWEEN® 60), polysorbate 20 (TWEEN® 20), N-dodecyl-βD-maltoside, Triton X-100, Brij 58, Poloxamer 188, Tyloxapol, PEG-40 stearate, NP-40, Pluronic™ F-68, and Poloxamer 4070. Each possibility is a separate embodiment of the invention. According to a certain embodiment, the nonionic surfactant is polysorbate, e.g., polysorbate 80 (TWEEN® 80).

According to additional embodiments, the water-soluble polymer is a synthetic water-soluble polymer having surface activity. According to further embodiments, the synthetic water-soluble polymer having surface activity is selected from the group consisting of polyvinyl alcohol, polypropylene oxide/ethylene oxide block co-polymers, copolymers of ethylene glycol/propylene glycol, poly-1,3-dioxolane, poly-1,3,6-trioxane, ethylene/maleic anhydride copolymer, propylene glycol homopolymers, and polyoxyethylated polyols. According to an exemplary embodiment, the water-soluble polymer having surface activity is polyvinyl alcohol.

According to additional embodiments, the pharmaceutical composition can further comprise a tonicity agent. According to a certain embodiment, the tonicity agent is sodium chloride.

According to further embodiments, the pharmaceutical composition can further comprise a buffering agent. Examples of buffering agents include, but are not limited to, phosphate buffer, acetate buffer, Tris buffer, and citrate buffer.

According to yet further embodiments, the pharmaceutical composition can further comprise a pharmaceutically acceptable carrier or diluent. According to a certain embodiment, the carrier is water.

According to still further embodiments, the pharmaceutical composition is formulated in the form selected from the group consisting of a solution, a suspension, an emulsion, powder, a tablet, and a capsule. According to a certain embodiment, the composition is formulated in the form of an aqueous solution. According to an exemplary embodiment, the pharmaceutical composition comprises GcMAF, polysorbate 80, sodium chloride, phosphate buffer, and water, wherein the pH of the composition ranges between about 5 to about 8, and wherein the concentration of GcMAF in the composition ranges from about 100 ng/ml to about 1 mg/ml, alternatively from about 100 ng/ml to about 300 μg/ml, further alternatively from about 200 ng/ml to about 30 μg/ml, and still further alternatively from about 200 ng/ml to about 1 μg/ml. Each possibility represents a separate embodiment of the invention.

According to another aspect, the present invention provides a method for treating a disease or disorder associated with macrophage activation, the method comprising administering to a subject in need of such treatment a therapeutically effective amount of the pharmaceutical composition according to the principles of the present invention.

According to some embodiments, the subject is a human being or an animal. According to a certain embodiment, the animal is a pet animal, such as a dog.

According to additional embodiments, the disease or disorder associated with macrophage activation is selected from the group consisting of cancer, viral diseases, bacterial infections, autoimmune diseases, autism, and chronic fatigue syndrome. Each possibility represents a separate embodiment of the present invention.

According to additional embodiments, the stable GcMAF is human GcMAF or animal GcMAF having independently an N-acetylgalactosamine group linked to an amino acid residue. According to a certain embodiment, the GcMAF is human GcMAF having an N-acetylgalactosamine group linked to an amino acid residue. According to further embodiments, the GcMAF comprises the amino acid sequence as set forth in any one of SEQ ID NOs: 1 to 3. Each possibility represents a separate embodiment of the present invention.

According to additional embodiments, the pharmaceutical composition is administered by parenteral or by oral administration route. According to a certain embodiment, the pharmaceutical composition is administered by intravenous, intramuscular or by subcutaneous administration route. Each possibility represents a separate embodiment of the present invention.

According to another aspect, the present invention provides a pharmaceutical composition according to the principles of the invention for use in treating a disease or disorder associated with macrophage activation.

These and other embodiments of the present invention will be better understood in relation to the description, examples and claims that follow.

### BRIEF DESCRIPTION OF THE FIGURES

FIGS. 1A-B show Western blot of GcMAF diluted in PBS in the presence or absence of human serum albumin (HSA), TWEEN® 80 or both. FIG. 1A shows Western blot analysis of GcMAF samples which were diluted in PBS alone or in PBS containing TWEEN® 80, HSA or both and thereafter loaded on a 4-12% polyacrylamide gel for immunoblotting. FIG. 1B shows Western blot analysis of GcMAF samples which were diluted in PBS alone or in PBS containing TWEEN® 80, HSA or both, incubated for one week at 4° C., and then loaded on a 4-12% polyacrylamide gel for immunoblotting. Gc protein diluted in PBS and subjected to immunoblotting immediately thereafter was used as a control.

FIGS. 2A-E show Western blot of GcMAF diluted in PBS in the absence or presence of arginine, HSA, TWEEN® 80, or mannitol or diluted in citrate buffer. The samples were either loaded immediately after dilution (FIG. 2A), or incubated for one week at 37° C. (FIG. 2B), two weeks at 25° C. (FIG. 2C), one month at 37° C. (FIG. 2D), or three months at 37° C. (FIG. 2E) prior to immunoblotting. As a control, a sample of Gc protein was diluted in PBS (FIGS. 2A-E) or in PBS containing TWEEN® 80 (FIG. 2E) and was immediately subjected to immunoblotting.

FIGS. 3A-B show Western blot of GcMAF diluted in PBS in the absence or presence of different concentration of TWEEN® 80. The samples were either loaded immediately after dilution (FIG. 3A), or incubated for one week at 37° C. (FIG. 3B) prior to immunoblotting. As a control, a sample of Gc protein was diluted in PBS (FIG. 3A) or in PBS containing TWEEN® 80 (FIGS. 3A and 3B) and was immediately subjected to immunoblotting.

FIGS. 4A-C show Western blot of GcMAF diluted in PBS in the absence or presence of the nonionic surfactants TWEEN® 80, TWEEN® 20, Poloxamer 188, and N-dodecyl-beta-D-maltoside. The samples were incubated overnight at 4° C. (FIG. 4A), one week at 37° C. (FIG. 4B), or one month at 37° C. (FIG. 4C) prior to immunoblotting. A sample of Gc protein diluted in PBS containing 0.005% TWEEN® 80 and subjected to immunoblotting immediately thereafter was used as a control.

FIGS. 5A-C show Western blot of GcMAF diluted in PBS in the absence or presence of TWEEN® 80, Brij® 58, or Triton X-100, PEG 4000, or trehalose. The samples were either loaded immediately after dilution (FIG. 5A) or incubated one week at 37° C. (FIG. 5B), or one month at 37° C. (FIG. 5C) prior to immunoblotting. A sample of Gc protein diluted in PBS containing 0.005% TWEEN® 80 and subjected to immunoblotting immediately thereafter was used as a control.

FIGS. 6A-B show Western blot of GcMAF diluted in PBS in the absence or presence of the nonionic surfactants TWEEN® 80, TWEEN® 20, Poloxamer 188, and N-dodecyl-beta-D-maltoside. The samples were either loaded immediately after dilution FIG. 6A) or incubated one month at 37° C. (FIG. 6B) prior to immunoblotting. A sample of Gc protein diluted in PBS containing 0.005% TWEEN® 80 and subjected to immunoblotting immediately thereafter was used as a control.

FIG. 7 shows Western blot of GcMAF after freezing and thawing. GcMAF diluted in PBS in the absence or presence of TWEEN® 80 was subjected to one cycle of freezing/thawing and then incubated at 4° C. or at room temperature (RT) for various periods of time prior to immunoblotting. As a control, samples of Gc protein were diluted in PBS or in PBS containing 0.005% TWEEN® 80 and immediately thereafter were subjected to immunoblotting.

FIG. 8 shows a Western blot of GcMAF after three cycles of freezing and thawing. GcMAF samples diluted in PBS in the absence or presence of TWEEN® 80 were subjected to three cycles of freezing and thawing, and thereafter to immunoblotting.

FIGS. 9A-C show Western blot of GcMAF diluted in PBS in the absence or presence of TWEEN® 80, hyaluronic acid, polyvinyl alcohol (PVA), alginate, casein, sodium dodecyl sulfate (SDS), or polyvinyl pyrolidone (PVP). The samples were either loaded immediately after dilution (FIG. 9A) or incubated for one week at 37° C. (FIG. 9B), or two weeks at 37° C. (FIG. 9C) prior to immunoblotting. As a control, a sample of Gc protein was diluted in PBS containing 0.005% TWEEN® 80 and immediately thereafter was subjected to immunoblotting.

FIGS. 10A-B show Western blot of GcMAF diluted in PBS in the absence or presence of TWEEN® 80, TWEEN® 20, Poloxamer 188, N-dodecyl-beta-D-maltoside, and polyvinyl alcohol (PVA). The samples were either loaded immediately after dilution (FIG. 10A) or incubated for one week at 37° C. (FIG. 10B). A sample of Gc protein diluted in PBS containing 0.005% TWEEN® 80 and subjected to immunoblotting immediately thereafter was used as a control.

### DETAILED DESCRIPTION OF THE INVENTION

The present invention provides stable pharmaceutical compositions comprising Gc derived macrophage activating factor (GcMAF) and a pharmaceutically acceptable excipient selected from the group consisting of pharmaceutically acceptable surfactants and pharmaceutically acceptable synthetic water-soluble polymers having surface activity. The present invention is based in part on the finding that storage of aqueous solutions of GcMAF at low concentrations results in significant loss of the chemical stability and biological activity of GcMAF when the solutions are stored at 4° C. Without being bound to any mechanism of action, the stabilizing effect of the surfactant may be due to prevention or reduction of GcMAF aggregation and/or degradation and/or absorption to the container's surfaces which may result from protein-surfactant direct interaction and binding of hydrophobic domains.

### Gc Protein and GcMAF

Gc protein, also designated vitamin D binding protein (DBP), is a plasma protein of an apparent molecular weight of 52 kDa with specific oligosaccharides attached thereto. Gc protein can be purified from blood serum or plasma by any method known to a person skilled in the art. Gc protein of high purity can be isolated from serum or plasma by 25-hydroxyvitamin D₃-Sepharose affinity chromatography. The Gc protein can also be purified by actin-agarose affinity chromatography which takes advantage of the binding specificity of the Gc protein for actin.

Alternatively, the Gc protein can be obtained from isolated cDNA encoding the Gc protein or the Gc protein small domain (domain III). Cloning and expression of the Gc protein and the Gc domain III was described in U.S. Pat. No. 6,410,269, incorporated by reference as if fully set forth herein. The method described therein employs a human liver cDNA library in bacteriophage λgt11 (Clontech, Palo Alto, Calif.) for isolating a full length cDNA encoding the human Gc protein, and the use of the baculoviral expression system in insect cells for the protein expression. However, mammalian cell systems are preferred for expressing a cDNA encoding the Gc protein or active fragment thereof. Preferably, expression is performed in eukaryotic cells such that the Gc protein or its active domain is correctly glycosylated. Any such cell system known in the art may be used, for example Chinese hamster ovary (CHO) cells, BHK cells, human embryonic kidney HEK293 cells and Saccharomyces cerevisiae. Accordingly, any eukaryotic expression vector can be used, including, but not limited to, pCI-NEO, pWE3, pcDNA3.1 and pCM182. Insertion of the vector into the selected cell system can be performed, for example, by electroporation, by lipid transfection such as TransFectin or by any chemical method known to a person skilled in art, with or without amplification. The transfection may result in transient or stable expression, both forms being adequate to obtain the desired Gc protein or fragment thereof. The expressed protein, being the precursor of active MAF, can then be extracted from the cells or collected from the growth media by any method known in the art.

Gc protein is a polymorphic protein which appears in two major phenotypes as demonstrable by gel electrophoresis analysis: Gc1 and Gc2. The entire nucleotide coding sequences of the Gc1 and Gc2 genes and the predicted amino acid sequences have been reported (Cook et al., 1985. J. Clin. Invest. 76: 2420; Yang et al., 1985. Proc. Natl. Acad. Sci. USA 82 7994). Gc1 is further divided into Gc1f and Gc1s subtypes, which migrate electrophoretically as two bands (“fast” and “slow”), due to a variation in one amino acid residue.

Gc1 protein is the major subtype of human Gc protein. It carries a branched trisaccharide composed of N-acetylgalactosamine (GalNAc) attached to the core protein with a termini of galactose and sialic acid (in Gc1f) or galactose and mannose or sialic acid (in Gc1s). Gc2 has a simple glycosylation pattern with a core GaINAc linked to a terminal galactose moiety. Gc1f oligosaccharide is hydrolyzed by membranous β-galactosidase of inflammation-primed B cells to yield a macrophage pro-activating factor, which is in turn hydrolyzed by sialidase (also known as neuraminidase) of T-cells to yield a macrophage activating factor (GcMAF). Animal, e.g., mouse or dog, DBP carries a disaccharide composed of N-acetylgalactosamine with a terminal galactose. Hydrolysis of this disaccharide by β-galactosidase of B cells alone generates a potent MAF (also designated GcMAF).

The terms “Gc protein” or “vitamin D-binding protein” as used herein refer to human or animal Gc protein, to all genotypes and polymorphic forms, including glycosylation forms, e.g., Gc1, Gc2, Gc1f, Gc1s and Gc1s*, and biologically active variants and fragments thereof. The term “biologically active” variant or fragment as used herein refers to any variant or fragment of Gc protein which upon deglycosylation produces GcMAF variant or fragment, the GcMAF variant or fragment thus produced is capable of activating macrophages and has an N-acetylgalactosamine group linked to an amino acid residue, typically to a threonine.

According to some embodiments of the present invention, the Gc protein comprises or consists of the amino acid sequence selected from the group consisting of SEQ ID NOs:1 to 3 (Gc1f, Gc1s and Gc2, respectively). The N-acetylgalactosamine group in these amino acid sequences is linked to threonine at positions 418 or 420 of the mature Gc protein.

The term “fragment” as used herein refers to any portion of the full length amino acid sequence of Gc protein which has less amino acids than the full length amino acid sequence of Gc protein, e.g., less than the 458 amino acids of Gc proteins of SEQ ID NOs:1 to 3, which portion still contains N-acetylgalactosamine group linked to an amino acid residue, typically to a threonine, and still retains macrophage activating activity. Typically, a portion of a full length protein is a peptide, polypeptide or protein. By “peptide” it is meant an amino acid sequence consisting of not more than 50 amino acids. By “polypeptide” it is meant an amino acid sequence generally consisting of more than 50 amino acid residues. By “protein” it is meant one or more covalently attached polypeptide chains. The terms peptide, polypeptide and protein are used interchangeable throughout the specification.

A Gc protein fragment may comprise the amino acid sequence corresponding to amino acids 400-435 of each of the mature Gc protein polymorphic forms. Alternatively, the Gc fragment can be the Gc protein domain III corresponding to amino acids 375-458 of the mature protein.

The Gc fragment Domain III, corresponding to amino acids 375-458 of the mature Gc protein, consists of the amino acid sequence as set forth in either SEQ ID NO:4 or SEQ ID NO:5. The N-acetylgalactosamine in these amino acid sequences is linked to threonine at positions 44 or 46.

The biologically active variants or fragments of the GC protein should retain the desired biological activities of the native polypeptide such that the variant or fragment polypeptide has the same therapeutic effect as the native polypeptide when administered to a subject. That is, the variant or fragment polypeptide will serve as a therapeutically active component in a pharmaceutical composition in a manner similar to that observed for the native polypeptide. The term “variant” refers to a variant of either the native Gc protein or of a fragment of the native Gc protein, wherein the variant comprises a fragment or a full length of the native polypeptide sequence having one or more amino acid substitutions, insertions, or deletions.

Biologically active variants of Gc protein will generally have at least 50%, preferably at least 60%, more preferably at least 70%, 80%, 90%, 95%, 98%, and most preferably about 99% sequence identity to the full length amino acid sequence of the Gc protein, which serves as the basis for comparison. Alternatively, the biologically active variants have at least 70%, 80%, 90%, 95%, 98%, and most preferably about 99% sequence identity to Domain III of the Gc protein, which serves as the basis for comparison. The term “sequence identity” refers to the same amino acid residues that are found within the variant polypeptide and the polypeptide molecule that serves as a reference when a specified, contiguous segment of the amino acid sequence of the variant is aligned and compared to the amino acid sequence of the reference molecule. The percentage sequence identity between two amino acid sequences is calculated by determining the number of positions at which the identical amino acid residue occurs in both sequences to yield the number of matched positions, dividing the number of matched positions by the total number of positions in the segment undergoing comparison to the reference molecule, and multiplying the result by 100 to yield the percentage of sequence identity.

When considering percentage of amino acid sequence identity, some amino acid residues may differ as a result of conservative amino acid substitutions, which do not affect the properties of the protein function. In these instances, percent sequence identity may be adjusted upwards to account for the similarity in conservatively substituted amino acids. Thus, substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are known as conservative substitutions.

Step-wise removal of certain oligosaccharides with specific glycosidase enzymes results in transforming the Gc protein to a highly potent macrophage activating factor (GcMAF) as disclosed herein above and in U.S. Pat. No. 5,177,002, incorporated by reference as if fully set forth herein. GcMAF can be further purified by ion exchange chromatography, e.g., anion exchange chromatography, and/or by hydrophobic chromatography, e.g., phenyl sepharose chromatography, as disclosed in WO 2012/137199, incorporated herein by reference.

### GcMAF Compositions

Stability of polypeptides at low concentrations in liquid formulations can be affected, for example, by factors such as the pH and/or ionic strength of the formulation, storage temperature, repeated cycles of freeze-thaw, adsorption to the container, and exposure to mechanical shear forces such as during processing. Aggregate formation and loss of biological activity can also occur as a result of physical agitation and interactions of polypeptide molecules in solution and at the liquid-air interfaces within storage vials. As a result of agitation the protein can aggregate, form particles, and ultimately precipitate with other adsorbed proteins.

The pharmaceutical compositions of the present invention can comprise as an excipient a pharmaceutically acceptable surfactant. The term “surfactant” (also known as surface-active agent) includes any agent that lowers the surface tension (or interfacial tension) between two liquids or between a liquid and a solid or between gas and liquid. Surfactants may act as detergents, wetting agents, emulsifiers, foaming agents, and/or dispersants. Surfactants may be emulsifiers that are linking oil and water in the composition in the form of emulsion. The surfactant used in the present invention includes any nonionic, anionic, cationic, zwitterionic, and amphoteric pharmaceutically acceptable surfactant which is useful in medicaments for human, or for animals.

Nonionic surfactants include, but are not limited to, sorbitan fatty acid esters, polyoxysorbitan fatty acid esters, polyoxyalkylene higher alcohol ethers, and polyoxyalkylene higher alcohol esters. Thus, nonionic surfactants include polyoxyethylene sorbitol esters such as polysorbate 80 (TWEEN® 80), polysorbate 60 (TWEEN® 60) and polysorbate 20 (TWEEN® 20), Tyloxapol; polyoxyethylene isooctylphenyl ethers such as Triton X-100, polyoxyethylene nonylphenyl ethers such as NP-40, polyoxyethylene dodecyl ethers such as Brij 58, octyl glucoside, and alkyl maltoside such as n-dodecyl-beta-D-maltoside; Poloxamer 4070; Poloxamer 188; and polyoxyl 40 stearate. Each possibility is a separate embodiment of the invention. TWEEN® and Poloxamer surfactants are preferred because they are FDA approved for human use.

Nonionic surfactants can also include, but are not limited to, fatty alcohol ethoxylates (alkylpolyethylene glycols); alkylphenol polyethylene glycols; alkylmercaptan polyethylene glycols; fatty amine ethoxylates (alkylaminopolyethylene glycols); fatty acid ethoxylates (acylpolyethylene glycols); polypropylene glycol ethoxylates (Pluronics™; e.g., Pluronic F-68); fatty acid alkylol amides, (fatty acid amide polyethylene glycols); N-alkyl-, N-alkoxypoly-hydroxy-fatty acid amide, sucrose esters; sorbitol esters and polyglycol ethers, polyoxyethylene-hydrogenated castor oil, fatty acid alkanolamide, sucrose fatty acid esters, glycerol mono, di- and trioctanoate. Each possibility is a separate embodiment of the invention.

Anionic surfactants include, but are not limited to, alkyl sulfates, olefin sulfates, ether sulfates, monoglyceride sulfates, alkyl sulfonates, aryl sulfonates, olefin sulfonates, alkyl sulfosuccinates, aryl sulfosuccinates, including sodium dodecyl sulphate (SDS), dioctyl sodium sulfosuccinate, dioctyl sodium sulfonate. Each possibility represents a separate embodiment of the invention.

Cationic surfactants include, but are not limited to, benzalkonium salts, polyoxyalkylene alkylamines, alkylamines, alkanolamine fatty acid esters, quaternary ammonium fatty acid esters, dialkyl ammonium salts, alkyl pyridinium salts including stearylamine, triethanolamine oleate, benzethonium chloride. Each possibility is a separate embodiment of the invention.

Amphoteric surfactants include, for example, imidazoline-based amphoteric surfactants (such as a 2-cocoyl-2-imidazoliniumhydroxide-1-carboxyethyloxy-2-sodium salt), betaine-based surfactants (such as alkyl betaine, amide betaine, and sulfo betaine), and acylmethyl taurine. Each possibility is a separate embodiment of the invention.

The surfactant is present in the pharmaceutical composition of the present invention in an amount of from about 0.001% to about 10% by weight of the total weight of the composition, alternatively in an amount of from about 0.001% to about 0.5% by weight, or in an amount of from about 0.001% to about 0.2% by weight, or in an amount of from about 0.001% to about 0.05% by weight of the total weight of the composition.

The term “about” used throughout the specification and claims refers to ±10% of the value indicated.

The composition of the present invention can comprise a water-soluble polymer. The water-soluble polymer can be a synthetic polymer having surface activity. The term “surface activity” refers to the activity of an agent to lower or eliminate the surface tension (or interfacial tension) between two liquids or between a liquid and a solid or between gas and liquid. The water-soluble polymers having surface activity include, but are not limited to, polyvinyl alcohol, polypropylene oxide/ethylene oxide co-polymer, copolymer of ethylene glycol/propylene glycol, poly-1,3-dioxolane, poly-1,3,6-trioxane, ethylene/maleic anhydride copolymer, polyamino acid (either homopolymers or random copolymers), poly(n-vinyl pyrolidone)polyethylene glycol, propylene glycol homopolymer, and polyoxyethylated polyol. Each possibility is a separate embodiment of the present invention. According to certain embodiments, the water-soluble polymer having surface activity is polyvinyl alcohol or a polypropylene oxide/ethylene oxide co-polymer.

The water-soluble polymer can also be a semi-synthetic polymer. Examples of semi-synthetic water-soluble polymers include water-soluble cellulose derivatives, e.g., carboxymethylcellulose or hydroxyethylcellulose.

The water-soluble polymer is present in the composition in the amount ranging from about 0.001% to about 5% by weight of the total weight of the composition, alternatively from about 0.001% to about 0.5% by weight, further alternatively from about 0.01% to about 0.2% by weight of the total weight of the composition.

The pharmaceutical composition of the present invention can further comprise a pharmaceutically acceptable carrier. The term “carrier” refers to a diluent or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions.

The pharmaceutical composition can further comprise agents for adjustment of tonicity including, but not limited to, sodium chloride or dextrose. Sodium chloride is used in order to keep the osmotic pressure of the pharmaceutical composition of the present invention suitable for an injectable preparation, especially in an amount of about 25 mM to about 300 mM.

The pharmaceutical composition can further comprise a buffering agent. The “buffering agent” means an agent(s) which is added to the composition in order to adjust the pH value in a solution preparation or in a lyophilized preparation when reconstituted. It is to be appreciated that pH of a liquid composition affects the stability of a polypeptide contained therein, primarily through its effect on polypeptide aggregate formation. Thus, the amount of a buffering agent present in the compositions of the invention depends upon the pH optimum for stability of GcMAF. Determination of this pH optimum can be achieved using methods generally available in the art.

Representative buffering agents that can be included in the compositions of the present invention are, for example, phosphate buffer, acetate buffer, Tris buffer and citrate buffer. Each possibility is a separate embodiment of the invention. The buffering agent adjusts the pH value of the solution so that the stability of GcMAF is maintained. The pH value of the present composition is preferably in the range of about 5 to about 8. The final concentration of the buffering agent in the composition is in the range of about 1 mM to about 100 mM.

The pharmaceutical composition can further comprise a stabilizer. As used herein, the term “stabilizer” refers to an excipient which maintains the chemical structure and/or biological activity of the GcMAF or any variant or fragment thereof of the present invention.

Stabilizers which are useful in practicing the present invention include, for example, amino acids such as arginine, lysine, aspartic acid, glutamic acid, or glycine or proteins such as casein or albumin. Alternatively, the stabilizer may be a sugar or a sugar alcohol. Any sugar such as mono-, di-, or polysaccharides, or water-soluble glucans, including for example trehalose, fructose, glucose, mannose, sorbose, xylose, maltose, lactose, sucrose, dextran, pullulan, dextrin, cyclodextrin, soluble starch, hydroxyethyl starch and cellulose derivatives, e.g., hydroxyethylcellulose and carboxymethylcellulose may be used. Sugar alcohol is defined as a C4-C8 hydrocarbon having an —OH group and includes, for example, mannitol, sorbitol, inositol, galacititol, dulcitol, xylitol, and arabitol. Each possibility is a separate embodiment of the invention. The sugars or sugar alcohols mentioned above may be used individually or in combination. Preferably, the sugar or sugar alcohol concentration is between about 1 w/v % and about 15 w/v %, more preferably between about 2 w/v % and about 10 w/v %. The stabilizer may also be a polyhydric alcohol such as glycerin or propylene glycol.

Additional stabilizers can be methionine, EDTA or one of its salts such as disodium salt; each is known to protect polypeptides against methionine oxidation.

The pharmaceutical composition of the present invention can be formulated as a liquid. The liquid composition can be stored as is or can be stored in a frozen state, or in a dried form for later reconstitution into a liquid form or other form suitable for administration to a subject. The term “dried form” refers to the liquid composition which is dried either by freeze drying (i.e., lyophilization), spray drying, or air drying. For example, GcMAF can be dissolved in a suitable aqueous solvent such as distilled water for injection, a buffer solution, a physiological saline solution, etc., and thereto a surfactant and/or water-soluble polymer, and optionally a buffering agent, a salt, and a stabilizer can be added, the solution thus obtained can be sterilized by filtration through a filter. Then, the solution can be stored at 4° C., or at −20° C., or can be lyophilized to give a lyophilized composition.

The pharmaceutical compositions of the present invention are “stable” or “stabilized” compositions. The terms “stable” or “stabilized” compositions refer to compositions having increased storage stability relative to compositions prepared in the absence of the surfactant and/or water-soluble polymer as disclosed herein. This increased storage stability is typically observed in the liquid formulation stored directly in that form for later use, although increased stability can also be obtained in a liquid formulation stored in a frozen state and thawed prior to use, or prepared in a dried form, such as a lyophilized, air-dried, or spray-dried form, for later reconstitution into a liquid form or other form prior to use. Preferably, the compositions of the invention are stored in their liquid form to take full advantage of the convenience of having increased storage stability in the liquid form, ease of administration without reconstitution, and ability to supply the formulation in prefilled, ready-to-use syringes or containers or as multidose preparations if the composition is compatible with bacteriostatic agents. The compositions of the present invention are preferably stable for at least one month at 2-8° C., alternatively for at least 2 months, 3 months, 4, 5, or at least 6 months at 2-8° C. Alternatively, the compositions of the invention are stable for at least one week at 37° C., or for at least two weeks, at least three weeks, at least four weeks, at least 2 months, or at least three months at 37° C. Each possibility is a separate embodiment of the invention. The stability of the composition of the present invention can be determined by measuring the protein content of GcMAF in a liquid composition comprising the surfactant and/or water-soluble polymer prior to storage, namely immediately after GcMAF production, and after storage. Thus, a stable composition is meant a liquid composition having at least 50% of the GcMAF protein amount after storage of one month at 2-8° C. as compared to the amount of GcMAF prior to storage, i.e., immediately after GcMAF production, alternatively of at least 60%, 70%, 80%, 90%, 95%, or at least 97% of the amount of GcMAF protein after storage of one month at 2-8° C. as compared to the amount of GcMAF prior to storage. Each possibility is a separate embodiment of the invention. GcMAF in the compositions of the present invention is “stable” or “stabilized”. The terms “stable” or “stabilized” GcMAF refer to GcMAF having increased storage stability, e.g., chemical stability, in the compositions comprising a surfactant and/or a water-soluble polymer as detailed herein relative to compositions devoid of the surfactant and/or water-soluble polymer.

The pharmaceutical composition, if desired, can also contain minor amounts of antibacterial agents such as benzyl alcohol or methyl parabens; antioxidants such as ascorbic acid or sodium bisulfite; and chelating agents such as ethylenediaminetetraacetic acid (EDTA).

For administration to a subject via the route chosen, such as by injection or infusion, the pharmaceutical composition must be safe, sterile, and must retain the desired therapeutic activity of GcMAF.

The pharmaceutical composition of the present invention can be formulated as a liquid, such as a solution, suspension, or emulsion, i.e., oil-in-water emulsion, water-in-oil emulsion, microemulsion or nanoemulsion. The pharmaceutical composition can be prepared in a dried form, such as a lyophilized powder, which can be reconstituted into a liquid solution, suspension, or emulsion before administration by any method including parenteral or oral routes of administration. The stabilized pharmaceutical composition is preferably sterilized by membrane filtration and is stored in unit-dose or multi-dose containers such as sealed vials or ampoules. Each possibility of formulation is a separate embodiment of the invention.

The pharmaceutical compositions can also take the form of tablets, capsules, sustained-release formulations and the like. Each possibility is a separate embodiment of the invention. The pharmaceutical composition can be formulated as a suppository, with traditional binders and carriers such as triglycerides, microcrystalline cellulose, gum tragacanth or gelatin.

Formulation for oral administration, e.g., tablets and capsules, can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of GcMAF, preferably in a substantially purified form, together with a suitable amount of a carrier so as to provide the form for proper administration to the subject.

### Uses of GcMAF

The present invention provides a method for treating a disease or disorder associated with macrophage activation comprising administrating to a subject in need of such treatment a therapeutically effective amount of a stable pharmaceutical composition according to the principles of the present invention.

According to one embodiment, the subject is a human being. According to additional embodiments, the subject is an animal, such as a domestic pet animal. According to a certain embodiment, the pet animal is a dog.

A “therapeutically effective amount” is that amount which is useful in the treatment or prevention of a disease or disorder associated with macrophage activation.

The pharmaceutical composition of the invention can be administered via a parenteral route of administration, such as by intravenous, intramuscular, subcutaneous, intraarterial and intraperitoneal injection or infusion. Alternatively, the pharmaceutical composition can be administered topically, nasally, or by oral administration. Each possibility is a separate embodiment of the invention. In an exemplary embodiment, the pharmaceutical composition can be administered by injection, preferably by intramuscular or subcutaneous injection.

It may be desirable to administer the pharmaceutical composition of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, by means of a medical patch or by means of an implant, said implant being of a porous, non-porous, or gelatinous material. According to some embodiments, administration can be by direct injection e.g., via a syringe, at the site of a tumor or neoplastic or pre-neoplastic tissue.

According to some embodiments, the GcMAF can be administered at a dose of about 50 ng/injection to about 1000 ng/injection

According to some embodiments, the disease or disorder associated with macrophage activation is selected from the group consisting of cancer, viral diseases, bacterial infections, autoimmune diseases, and chronic fatigue syndrome.

According to further embodiments, the cancer to be treated by the pharmaceutical compositions of the present invention includes, but does not limited to, solid tumors such as sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, malignant synovioma, mesothelioma, Ewing's tumor leiomydsarcoma, rhabdomyosarcoma, colon carcinoma, pancreatic cancer, breast cancer, ovarian cancer, prostate cancer, squamous cell carcinoma, basal cell carcinoma, adenocarcinoma sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, oligodendroglioma, melanoma, neuroblastoma, and retinoblastoma. Each possibility is a separate embodiment of the invention. According to additional embodiments, the cancer to be treated is non-solid tumor such as leukemia.

According to further embodiments, the viral diseases to be treated include diseases associated with viruses such as, for example, human immunodeficiency viruses 1 and 2 (HIV-1 and HIV-2) including drug resistant strains, human T-cell leukemia viruses 1 and 2 (HTLV-1 and HTLV-2), respiratory syncytial virus (RSV), human papilloma virus (HPV), adenovirus, hepatitis B virus (HBV), hepatitis C virus (HCV), Epstein-Ban-virus (EBV), varicella zoster virus (VZV), cytomegalovirus (CMV), herpes simplex viruses 1 and 2 (HSV-1 and HSV-2), human herpes virus 8 (HHV-8, also known as Kaposi's sarcoma-associated virus), and flaviviruses, including Yellow Fever virus, Dengue virus, Japanese Encephalitis and West Nile viruses, influenza virus. Each possibility is a separate embodiment of the invention.

According to yet further embodiments, the bacterial infections include an infection caused by bacteria selected from the group consisting of staphylococci, including Staphylococcus aureus (methicillin-resistant and -susceptible), Staphylococcus epidermidis, Staphylococcus hemolyticus, Staphylococcus saprophyticus, Staphylococcus lugdunensis, Staphylococcus capitis, Staphylococcus caprae, Staphylococcus saccharolyticus, Staphylococcus simulans, Staphylococcus warned, Staphylococcus hominis, Staphylococcus intermedius, Staphylococcus pseudointermedius, and Staphylococcus lyricus; streptococci, including Streptococcus pyogenes, Streptococcus agalactiae, Streptococcus dysgalactiae subspecies dysgalactiae, Streptococcus anginosus, Streptococcus mitis, Streptococcus salivarius, Streptococcus bovis, and Streptococcus mutans; enterococci, including Enterococcus faecalis and Enterococcus faecium; Nesseria species, including Neisseria gonorrhoeae, and Neisseria meningitides; Clostridium species, including Clostridium difficile; Bordetella species, including Bordetella pertussis; Bacillus species, including Bacillus anthracis; and Corynebacterium species, including Corynebacterium diphtheriae. Each possibility is a separate embodiment of the invention.

According to additional embodiments, the autoimmune disease is selected from the group consisting of rheumatoid arthritis, ankylosing spondylitis, Crohn's disease, psoriasis, Systemic Lupus Erythematosus (SLE), eczema, gout, inflammatory bowel disease, and multiple sclerosis. Each possibility is a separate embodiment of the invention.

The pharmaceutical composition of the present invention can also be useful for treating autism.

The following example is presented in order to more fully illustrate some embodiments of the invention. It should, in no way be construed, however, as limiting the broad scope of the invention. One skilled in the art can readily devise many variations and modifications of the principles disclosed herein without departing from the scope of the invention.

### Example 1

### Effect of Polysorbitan (POLYSOBATE 80) and Human Serum Albumin on GcMAF Stability

In order to determine the stability of GcMAF in solution for various periods of times, several stabilizing agents were added to the GcMAF solution, and the amount of GcMAF was evaluated.

GcMAF was prepared as previously described (Yamamoto et al., 2008, Cancer Immunol. Immunother. 57: 1007-1016). Briefly, Gc protein was purified from human serum or plasma using 25-hydroxyvitamin D3-affinity chromatography. The purified Gc was incubated sequentially with immobilized β-galactosidase and sialidase to form GcMAF. The GcMAF was filtered through a 0.22 micron filter for sterilization, and then diluted to a final concentration of 200 ng/ml in PBS pH 7.5 containing one of the following additives: 0.2% polysorbate 80 (TWEEN® 80), 600 ng/ml human serum albumin (HSA) or both. The solutions were filtered and aseptically filled in 1 ml aliquots into 2 ml glass vials with rubber stoppers and aluminum caps and kept at 4° C. Detection of Gc or GcMAF was performed by western blot analysis at time 0 and after 1 week by loading a total amount of 2.6 ng Gc or GcMAF per lane on 4-12% polyacrylamide gels. The gels were electrophoresed and transferred to nitrocellulose (NC) membranes which were probed with a polyclonal rabbit anti-Gc antibody followed by goat anti-rabbit antibody conjugated to alkaline phosphatase and chemiluminescent detection.

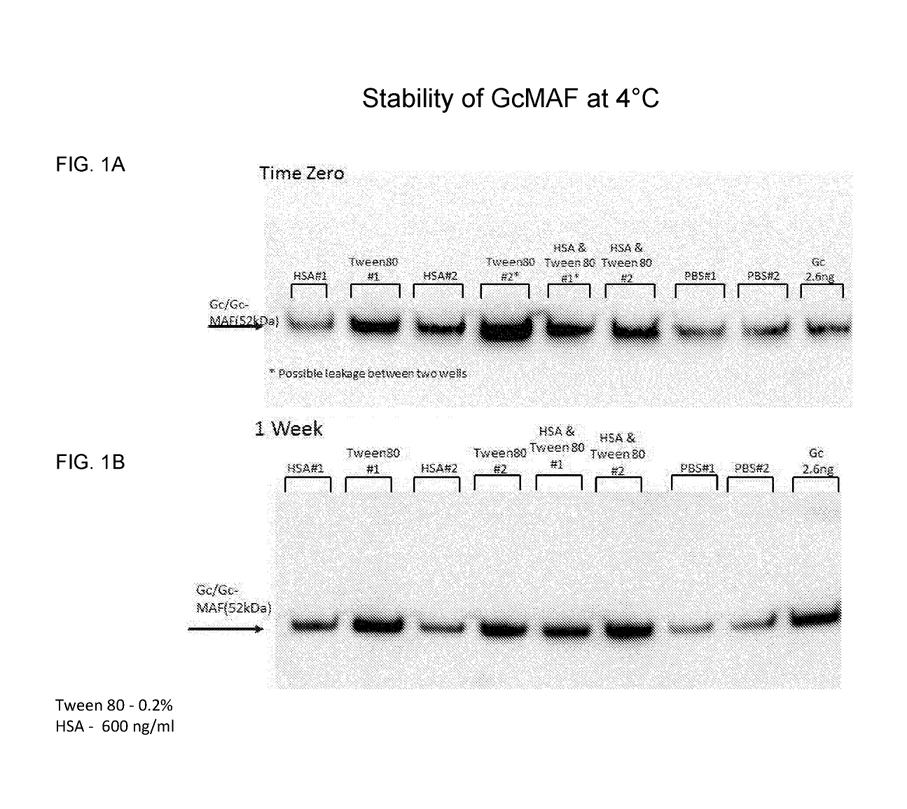

FIG. 1A shows western blot analysis performed at time point 0 (day of preparing the diluted samples). As shown in the figure, GcMAF is detected in all samples, however, samples diluted in the presence of TWEEN® 80 showed stronger band intensity than samples diluted in PBS only or in PBS and HSA. Commercial Gc protein (Calbiochem) diluted in PBS was used as a control and showed similar intensity as GcMAF diluted in PBS.

FIG. 1B shows western blot analysis of GcMAF or Gc samples which were left at 4° C. for 1 week. GcMAF samples containing TWEEN® 80 showed strong band intensity similar to that obtained by the freshly diluted commercial Gc protein. GcMAF samples diluted in PBS showed faint intensity compared to that obtained by GcMAF samples diluted in PBS containing TWEEN® 80. GcMAF samples diluted in PBS containing HSA showed stronger intensity than GcMAF samples diluted in PBS only, but fainter than the GcMAF samples containing TWEEN® 80. Thus, after one week of storage at 4° C., TWEEN® 80 at a concentration of 0.2% was found to maintain GcMAF in solution, and this nonionic surfactant was shown to be more effective than HSA in preserving GcMAF stability.

When GcMAF samples were stored in polypropylene vials, similar band intensity was obtained.

### Example 2

### Stability of GcMAF at Different Temperatures for Different Periods of Time

GcMAF was prepared as described in Example 1 herein above. Briefly, Gc protein was purified from human serum or plasma using 25-hydroxyvitamin D3-affinity chromatography. The purified Gc protein was incubated sequentially with immobilized β-galactosidase and sialidase to form GcMAF. The GcMAF was filtered through a 0.22 micron filter for sterilization. It was then diluted to a final concentration of 200 ng/ml in different buffer solutions: PBS pH 7.5 or citrate buffer pH 5.5. The PBS solution also contained one of the following additives: 0.01% TWEEN® 80, 600 ng HSA, or 50 μM arginine. All solutions were prepared in the presence or absence of 2% mannitol. The solutions were then filtered and aseptically filled in 1 ml aliquots into 2 ml glass vials with rubber stoppers and aluminum caps and kept at different temperatures (4° C., 25° C. and 37° C.). The samples were analyzed at different time periods by western blot analysis, loading a total amount of 2.6 ng of Gc protein or GcMAF per lane on 4-12% polyacrylamide gels. The gels were electrophoresed and transferred to NC membranes which were probed with a polyclonal rabbit anti-Gc antibody followed by goat anti-rabbit antibody conjugated to alkaline phosphatase and chemiluminescent detection.

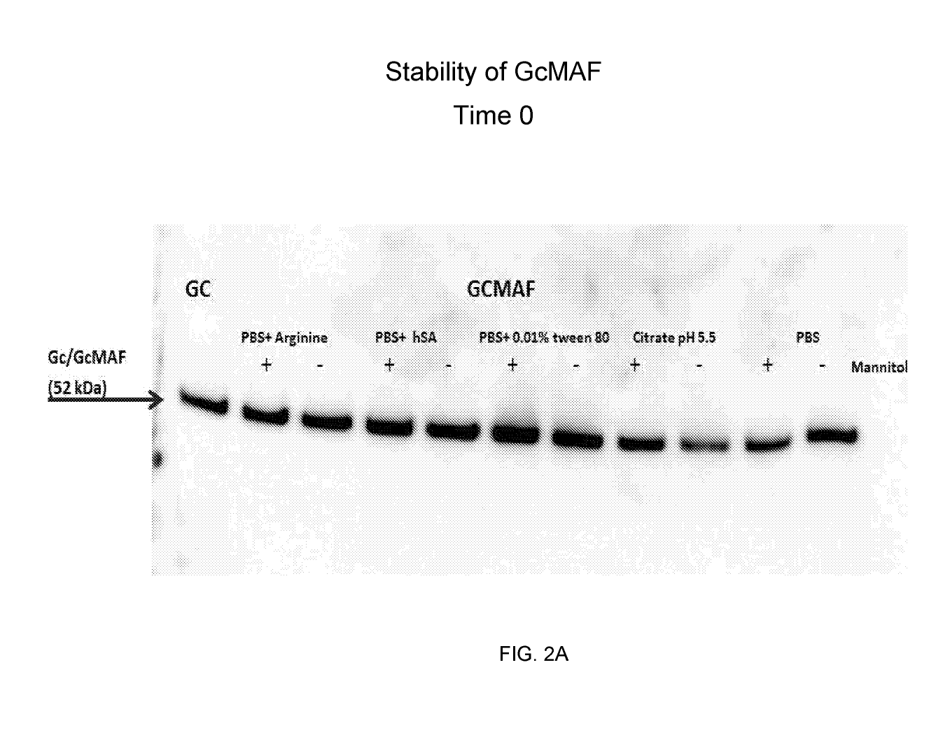

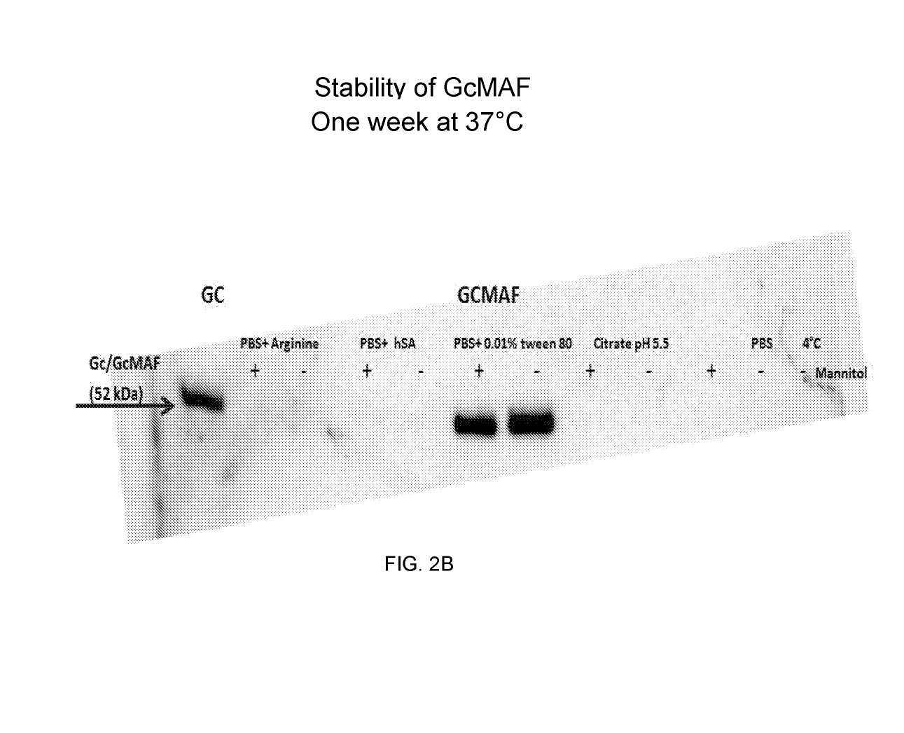

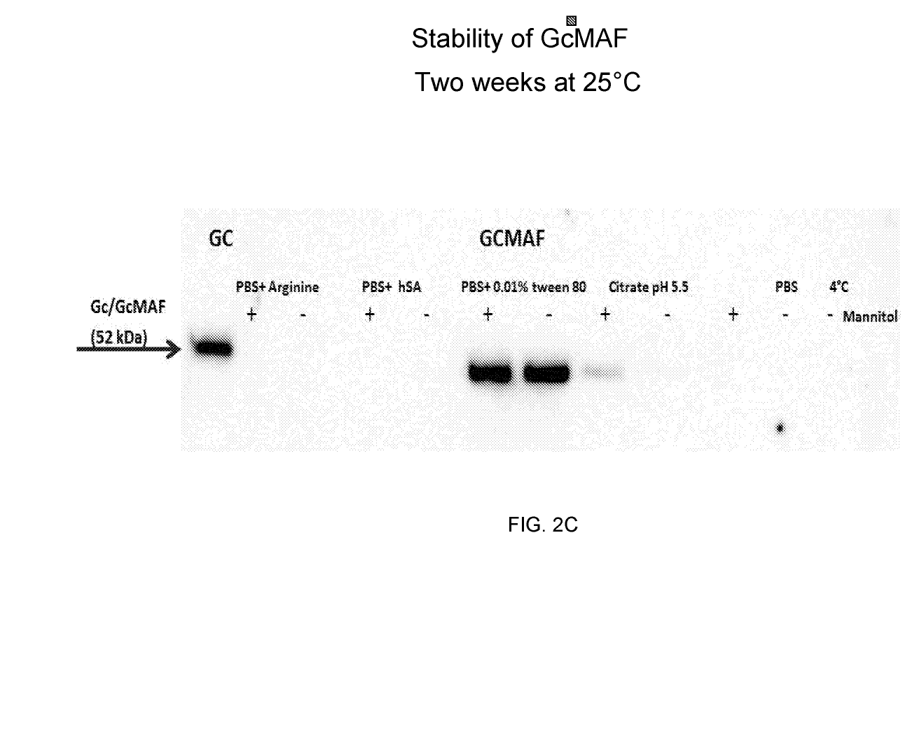

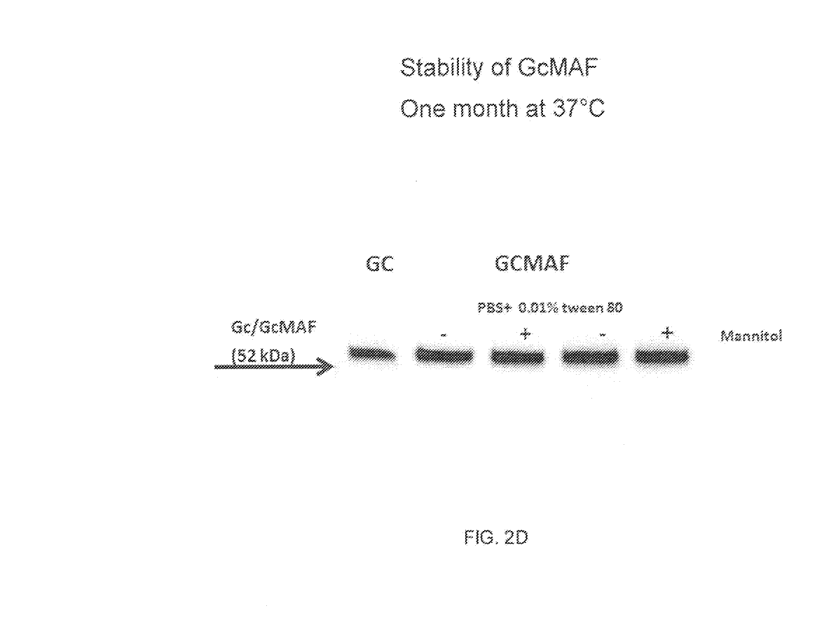

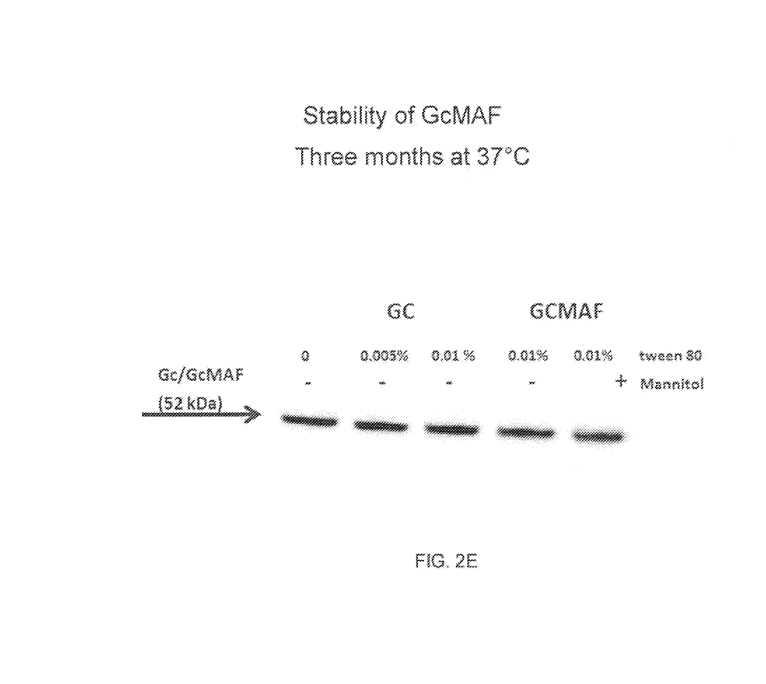

FIG. 2A shows western blot analysis performed at time point 0 (day of preparing the diluted samples). All GcMAF samples showed visible bands of a similar intensity as compared to the standard commercial Gc protein (Calbiochem).

FIG. 2B shows western blot analysis of GcMAF samples incubated for 1 week at 37° C. A GcMAF sample incubated at 4° C. in PBS only was also evaluated. As shown in the figure, strong intensity was observed when GcMAF samples were diluted in PBS containing TWEEN® 80. The intensity observed for these GcMAF samples was similar to that obtained by the freshly diluted Gc sample. Human serum albumin (HSA) was shown to be less active than TWEEN® 80 in maintaining GcMAF stability.

FIG. 2C shows western blot analysis of GcMAF samples incubated for 2 weeks at 25° C. A GcMAF sample incubated at 4° C. in PBS only was also evaluated. As shown in the figure, strong intensity was observed when GcMAF samples were diluted in PBS containing TWEEN® 80. The intensity observed for these GcMAF samples was similar to that obtained by the freshly diluted commercial Gc sample. Human serum albumin (HSA) was shown to be less active than TWEEN® 80 in maintaining GcMAF stability.

FIGS. 2D and 2E show western blot analysis of GcMAF samples containing TWEEN 80 incubated for one month (FIG. 2D) or for three months (FIG. 2E) at 37° C. as compared to a freshly diluted Gc sample. As shown in the figures, GcMAF samples incubated in the presence of TWEEN® 80 showed high intensity similar to that observed for the freshly diluted Gc protein. The high band intensity was obtained even after three months of incubation at 37° C. (roughly equivalent to 24 months at 4° C.).

ELISA was performed by adsorbing Gc or GcMAF samples to 96 well plates, probing with a rabbit polyclonal anti Gc antibody followed by HRP conjugated anti rabbit Goat antibody and detecting by absorbance. The absorbance of the samples containing 0.01% TWEEN® 80 that were incubated for 3.5 months at 37° C. was the same as the absorbance of a freshly diluted Gc protein (210 ng/ml).

Bioactivity test was performed by measuring the release of hydrogen peroxide (H₂O₂) in RAW264.7 mouse macrophage cell line to determine macrophage activation following stimulation with GcMAF (0.2 ng/0). Values of 2 folds and higher relative to controls without GcMAF were considered active. Release of hydrogen peroxide (>2 fold) was observed in GcMAF samples containing TWEEN® 80 (0.01%). that were incubated for 3.5 months at 37° C.

### Example 3

### Effect of Different Concentrations of TWEEN® 80 on GcMAF Stability

GcMAF was prepared as described in Example 1 herein above. Briefly, Gc protein was purified from serum or plasma using 25-hydroxyvitamin D3-affinity chromatography. The purified Gc protein was incubated sequentially with immobilized β-galactosidase and sialidase to form GcMAF. The GcMAF was filtered through a 0.22 micron filter for sterilization. It was then diluted to a final concentration of 200 ng/ml in PBS pH 7.5 containing different concentrations of the nonionic detergent TWEEN® 80 ranging from 0 to 0.01%. The solutions were filtered and aseptically filled in 1 ml aliquots into 2 ml glass vials with rubber stoppers and aluminum caps and were kept at 37° C. The samples were analyzed at different time points by western blot analysis, loading a total of 2.6 ng of Gc or GcMAF per lane on 4-12% polyacrylamide gels. The gels were electrophoresed and transferred to NC membranes which were probed with a polyclonal rabbit anti-Gc antibody followed by goat anti-rabbit antibody conjugated to alkaline phosphatase and chemiluminescent detection.

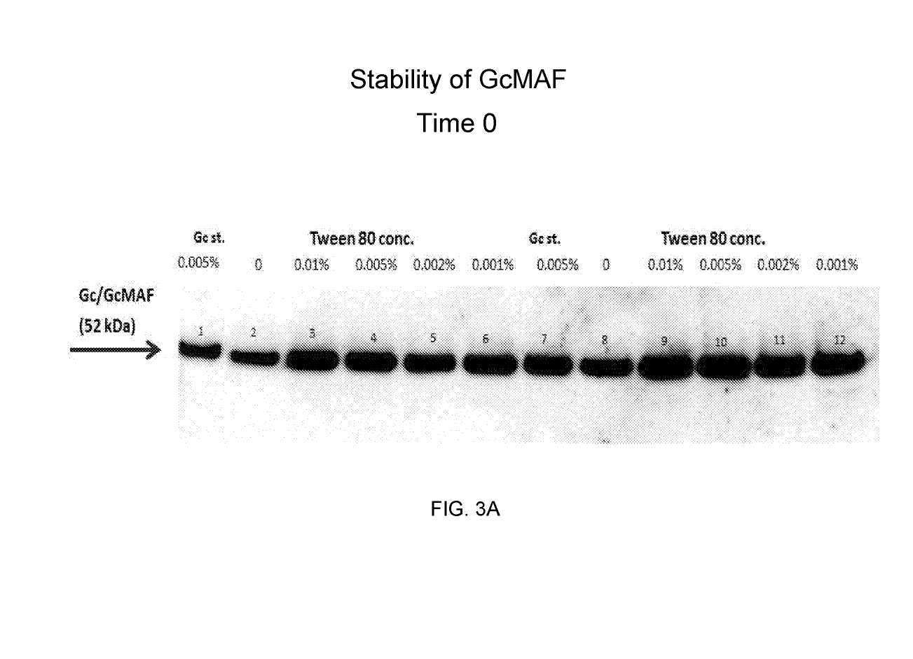

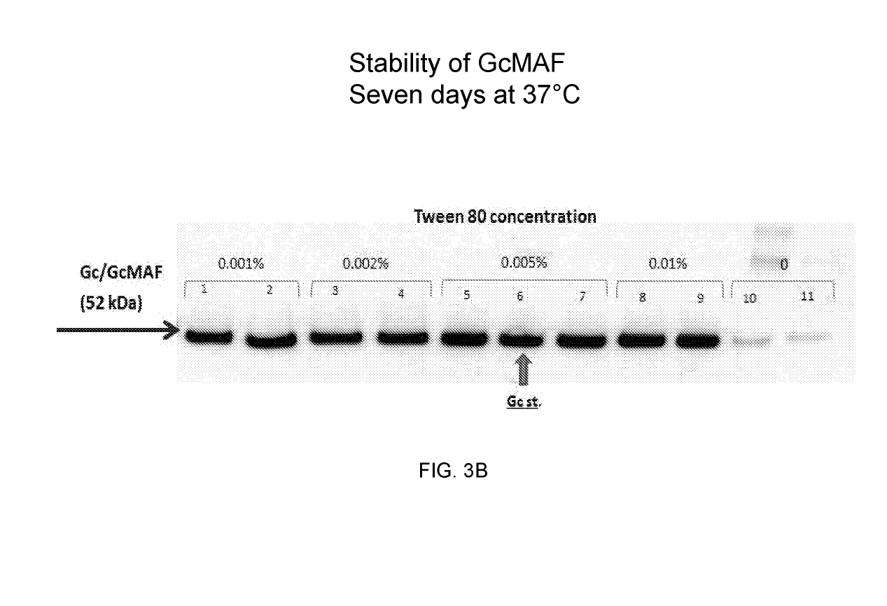

FIG. 3A shows western blot analysis performed at time point 0 (day of preparing the diluted samples). All samples showed similar intensity as compared to the standard commercial Gc protein (Calbiochem).

FIG. 3B shows western blot analysis of GcMAF or Gc samples incubated for one week at 37° C. GcMAF samples containing TWEEN® 80 (even at a concentration of 0.001%) showed clear visible bands with similar intensity to that obtained by the freshly diluted Gc protein. GcMAF samples which were prepared in PBS only (in the absence of detergents) showed very faint intensity compared to the standard Gc protein. Thus, TWEEN® 80 at concentrations of 0.001% or higher is highly effective in maintaining GcMAF in solution, and hence in preserving its stability.

### Example 4

### Effect of Various Nonionic Detergents on GcMAF Stability

GcMAF was prepared as described in Example 1 herein above. Briefly, Gc protein was purified from serum or plasma using 25-hydroxyvitamin D3-affinity chromatography. The purified Gc protein was incubated sequentially with immobilized β-galactosidase and sialidase to form GcMAF. The GcMAF was filtered through a 0.22 micron filter for sterilization. It was then diluted to a final concentration of 200 ng/ml in PBS pH 7.5 in the absence or presence the following nonionic surfactants at the indicated concentrations: TWEEN® 80 (0.005%), TWEEN® 20 (0.005%, 0.01% or 0.02%), Poloxamer 188 (0.02%, 0.1% or 0.2%) and N-dodecyl-beta-D-maltoside (0.01%, 0.02% or 0.05%). The solutions were filtered and aseptically filled in 1 ml aliquots into 2 ml glass vials with rubber stoppers and aluminum caps and were kept at 37° C. at an upside down position or at 4° C. The samples (in duplicates) were analyzed at different time points by western blot analysis, loading a total of 2.6 ng of Gc or GcMAF per lane on 4-12% polyacrylamide gels. The gels were electrophoresed and transferred to NC membranes which were probed with a polyclonal rabbit anti-Gc antibody followed by goat anti-rabbit antibody conjugated to alkaline phosphatase and chemiluminescent detection.

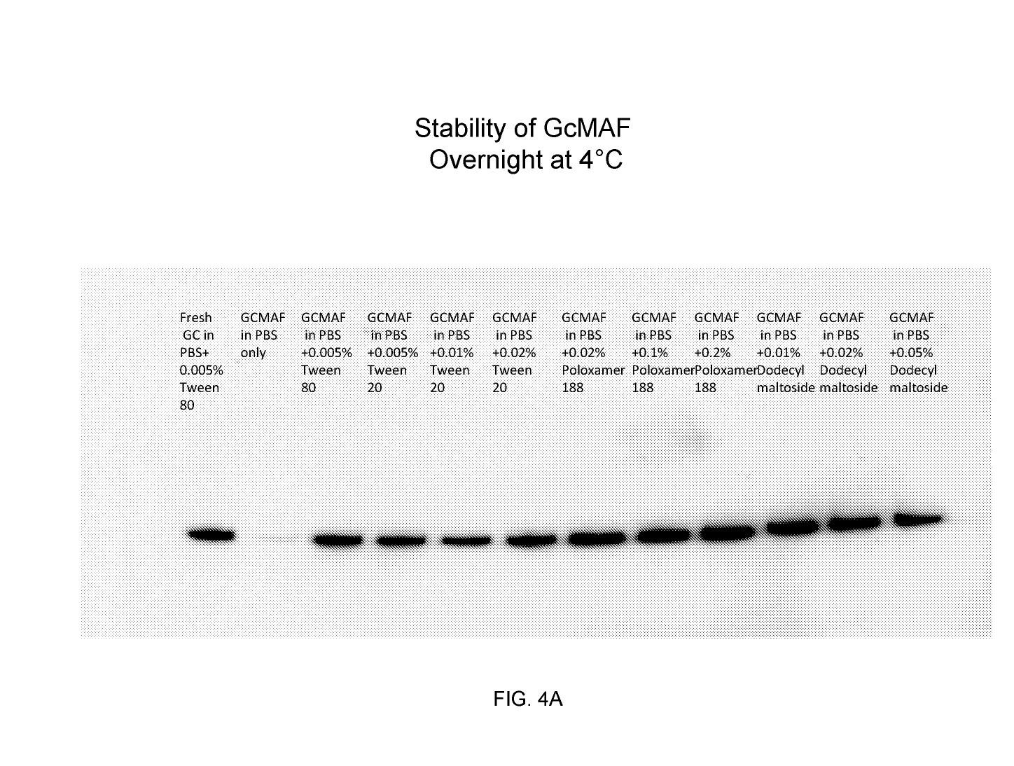

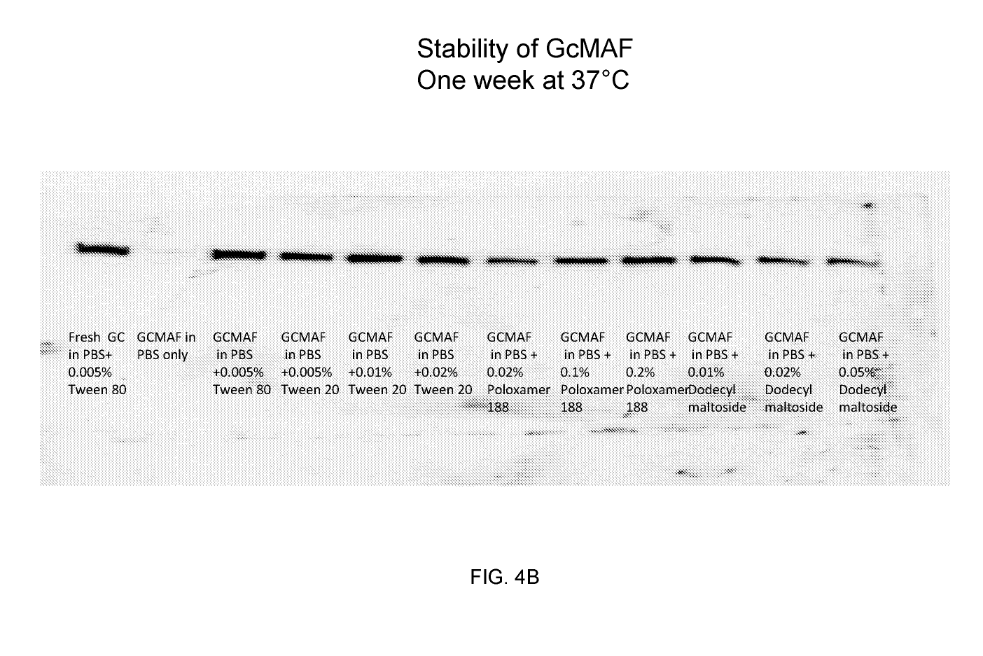

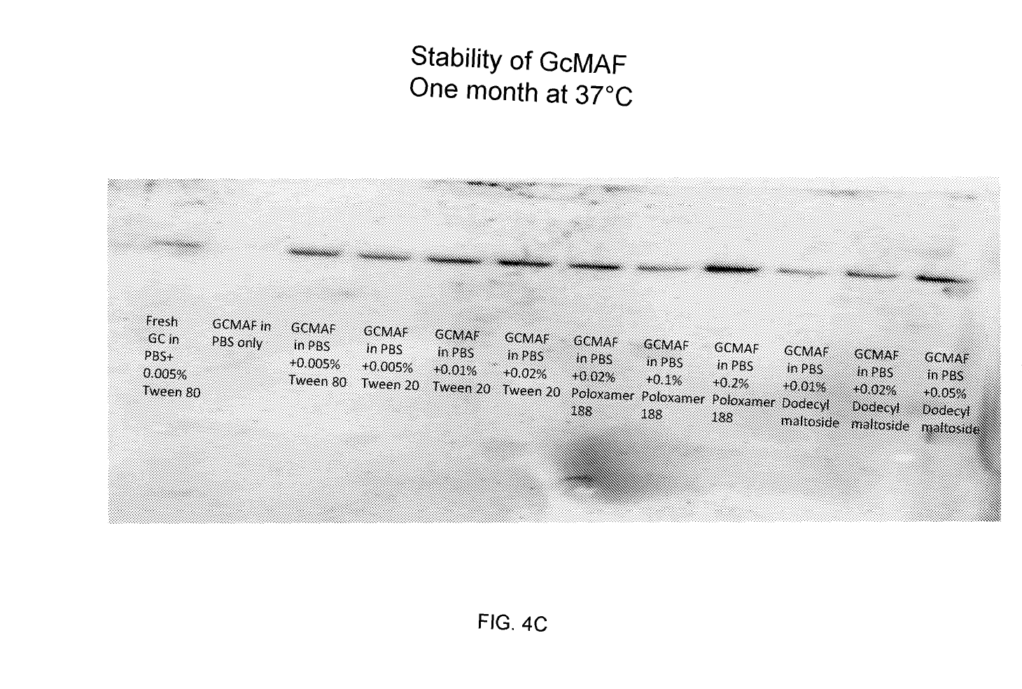

FIG. 4A shows western blot analysis performed after overnight incubation at 4° C. All samples showed similar staining intensity as compared to the standard commercial Gc protein (Calbiochem), except of the sample incubated in the absence of surfactants which was almost undetectable after overnight at 4° C.

FIG. 4B shows western blot analysis of GcMAF or Gc samples incubated for one week at 37° C. GcMAF samples incubated in the presence of the nonionic surfactants (at all of the tested concentrations) showed similar staining intensity to that obtained by the freshly diluted Gc protein. GcMAF samples prepared in PBS only and incubated in the absence of surfactants were not detected under these conditions.

FIG. 4C shows western blot analysis of GcMAF or Gc samples incubated for one month at 37° C. GcMAF samples incubated in the presence of the nonionic surfactants (at all of the concentrations tested) showed clear visible bands with similar staining intensity to that obtained by the freshly diluted Gc protein. GcMAF samples which were incubated in PBS only (in the absence of surfactants) were not visible. Thus, nonionic surfactants such as polysorbate 20, N-dodecyl-beta-D-maltoside or Poloxamer 188, are highly effective in maintaining GcMAF in solution even when stored for one month at 37° C., and hence these nonionic surfactants are useful in preserving GcMAF stability.

### Example 5

### Effect of Nonionic Surfactants and Other Stabilizers on GcMAF Stability

GcMAF was prepared as described in Example 1 herein above. Briefly, Gc protein was purified from serum or plasma using 25-hydroxyvitamin D3-affinity chromatography. The purified Gc protein was incubated sequentially with immobilized β-galactosidase and sialidase to form GcMAF. The GcMAF was filtered through a 0.22 micron filter for sterilization. It was then diluted to a final concentration of 200 ng/ml in PBS pH 7.5 in the absence or presence of the following nonionic surfactants at the indicated concentrations: TWEEN® 80 (0.005%), Triton X100 (0.06% or 0.12%.), and Brij® 58 (0.015% or 0.05%), or in the presence of the stabilizers Trehalose (1% or 2%) and PEG 4000 (1% or 3%). The solutions were filtered and aseptically filled in 1 ml aliquots into 2 ml glass vials with rubber stoppers and aluminum caps and were kept at 37° C. at an upside down position. The samples (in duplicates) were analyzed at different time points by western blot analysis, loading a total of 2.6 ng of Gc or GcMAF per lane on 4-12% polyacrylamide gels. The gels were electrophoresed and transferred to NC membranes which were probed with a polyclonal rabbit anti-Gc antibody followed by goat anti-rabbit antibody conjugated to alkaline phosphatase and chemiluminescent detection.

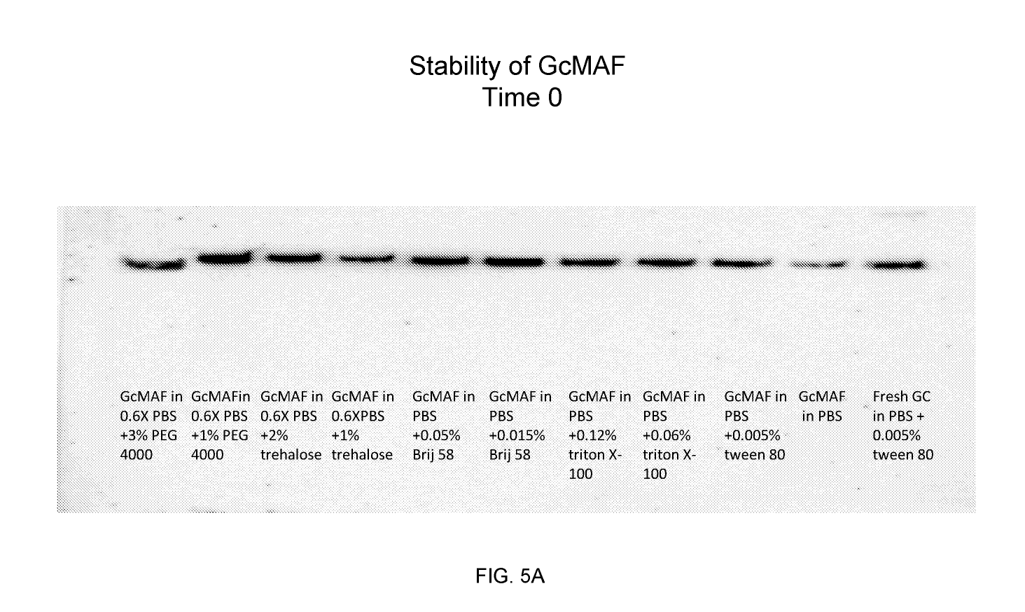

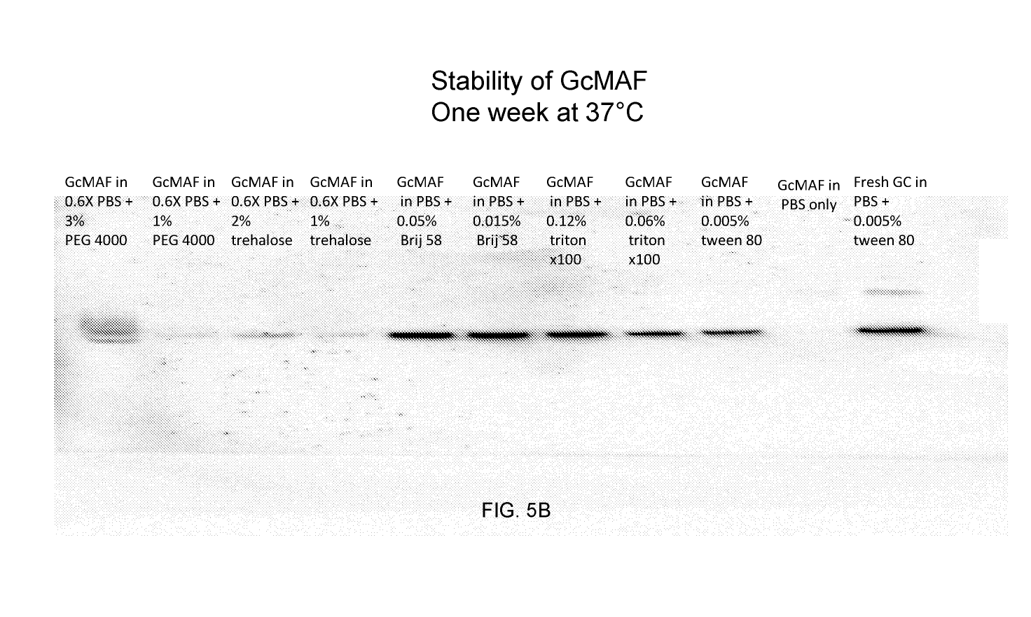

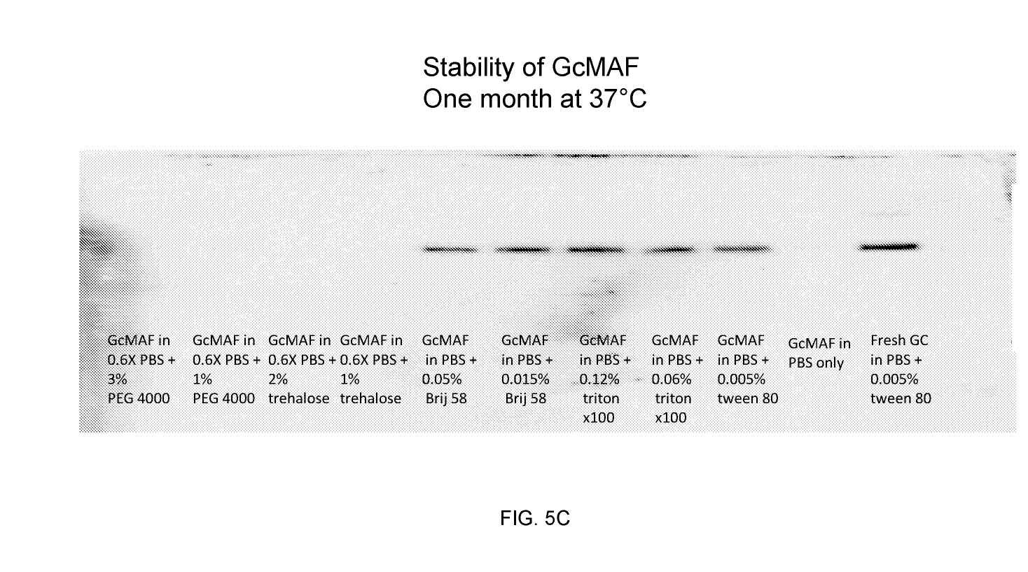

FIG. 5A shows western blot analysis performed at time point 0 (day of preparing the diluted samples). All samples showed similar staining intensity as compared to the standard commercial Gc protein (Calbiochem), except of the sample which was incubated in the absence of detergents and which was slightly fainter.

FIG. 5B shows western blot analysis of GcMAF or Gc samples incubated for one week at 37° C. GcMAF samples incubated in the presence of the nonionic surfactants (at all of the concentrations tested) showed clear visible bands with similar intensity to that obtained by the freshly diluted Gc protein. GcMAF samples which were incubated in PBS only (in the absence of surfactants) were not detectable, and samples incubated in the presence of Trehalose or PEG 4000 were almost non-detectable.

FIG. 5C shows western blot analysis of GcMAF or Gc samples incubated for one month at 37° C. GcMAF samples incubated in the presence of the nonionic surfactants (at all of the concentrations tested) showed clear visible bands with only slightly fainter intensity compared to that obtained by the freshly diluted Gc protein. GcMAF samples which were incubated in PBS only (in the absence of surfactants) or with Trehalose or PEG 4000 at all the concentrations tested were not detectable under these experimental conditions. Thus, nonionic surfactants, e.g., Triton X-100 and Brij 58, were found to maintain GcMAF stability even after one month of storage at 37° C. Trehalose or PEG 4000 were found to be less effective.

### Example 6

### Effect of Nonionic Surfactants on the Stability of GcMAF at Higher Concentration

GcMAF was prepared as described in Example 1 herein above. Briefly, Gc protein was purified from serum or plasma using 25-hydroxyvitamin D3-affinity chromatography. The purified Gc protein was incubated sequentially with immobilized β-galactosidase and sialidase to form GcMAF. The GcMAF was filtered through a 0.22 micron filter for sterilization. It was then diluted to a final concentration of 2.5 μg/ml in PBS pH 7.5 containing different concentrations of the nonionic surfactants: TWEEN® 80 (0.005%), TWEEN® 20 (0.005%), Poloxamer 188 (0.05%) and dodecyl maltoside (0.01%). The solutions were filtered and aseptically filled in 1 ml aliquots into 2 ml glass vials with rubber stoppers and aluminum caps and were kept at 37° C. at an upside down position. The samples (in duplicates) were analyzed at different time points by ELISA, and western blot analysis, loading a total of 5 ng of Gc or GcMAF per lane on 4-12% polyacrylamide gels. The gels were electrophoresed and transferred to NC membranes which were probed with a polyclonal rabbit anti-Gc antibody followed by goat anti-rabbit antibody conjugated to alkaline phosphatase and chemiluminescent detection.

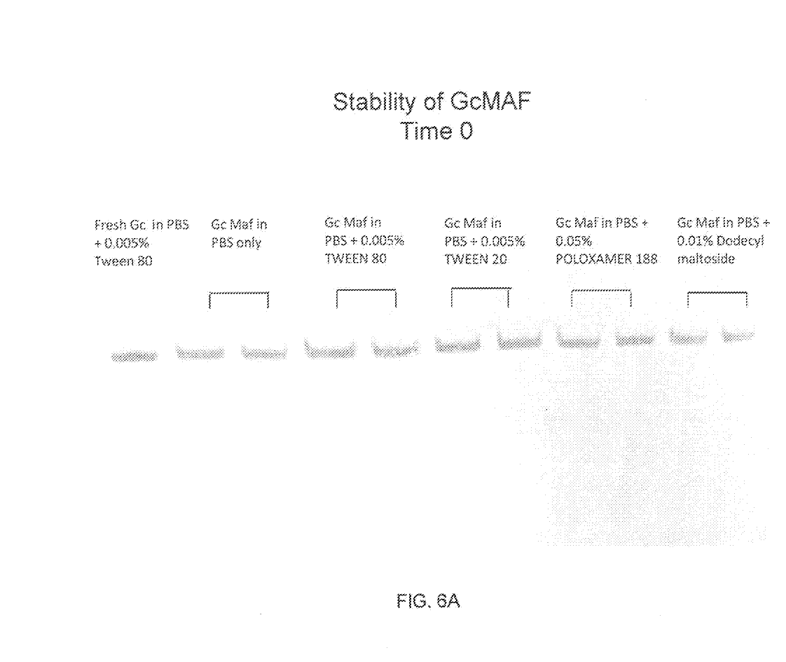

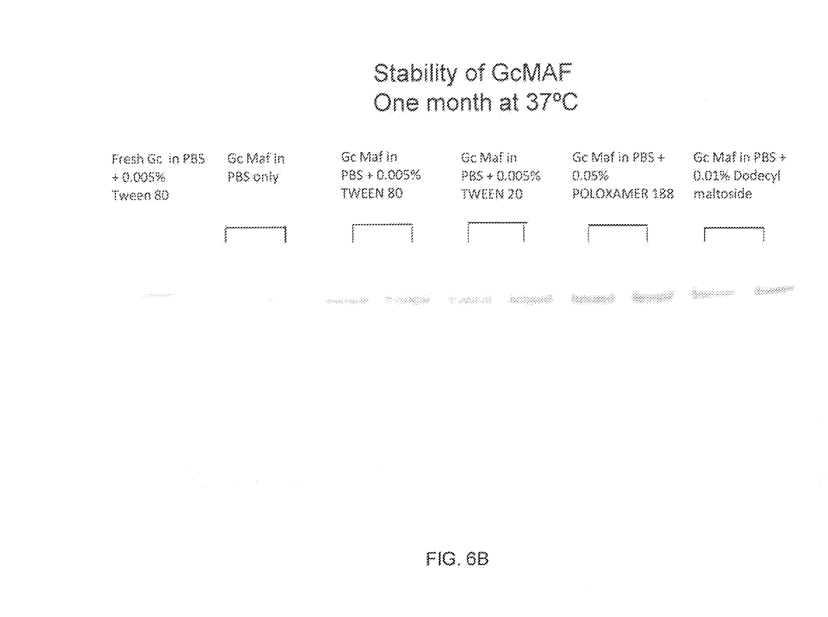

FIG. 6A shows western blot analysis performed at time point 0 (immediately after preparing the samples). All samples showed similar staining intensity as compared to the standard commercial Gc protein (Calbiochem).

FIG. 6B. shows western blot analysis of GcMAF or Gc samples incubated for one month at 37° C. GcMAF samples incubated in the presence of the nonionic surfactants showed clear visible bands with similar intensity to that obtained by the freshly diluted Gc protein. GcMAF samples which were incubated in PBS only (in the absence of surfactants) showed significantly weaker staining compared to the freshly prepared standard GC or to the GcMAF samples containing surfactants.

ELISA was performed by adsorbing Gc or GcMAF samples to 96 well plates, probing with a rabbit polyclonal anti Gc antibody followed by HRP conjugated anti rabbit Goat antibody and detecting by absorbance. Samples of GcMAF incubated for 1 month at 37° C. in the presence of the nonionic surfactants showed similar absorbance as of the samples at time 0. GcMAF incubated in PBS in the absence of surfactants showed significant decrease in concentration (39% of the signal at time 0).

### Example 7

### Effect of Time and Storage Temperature on GcMAF Stability after its Thawing in the Presence or Absence of Nonionic Surfactants

GcMAF was prepared as described in Example 1 herein above. Briefly, Gc protein was purified from serum or plasma using 25-hydroxyvitamin D3-affinity chromatography. The purified Gc protein was incubated sequentially with immobilized β-galactosidase and sialidase to form GcMAF. The GcMAF was filtered through a 0.22 micron filter for sterilization. It was then diluted to a final concentration of 200 ng/ml in PBS pH 7.5 or in PBS containing 0.005% TWEEN® 80. The solutions were filtered and aseptically filled in 1 ml aliquots into 2 ml glass vials with rubber stoppers and aluminum caps and were frozen at −20° C. The samples (in duplicates) were thawed and kept at RT or at 4° C. up to overnight and analyzed at different time points by western blot analysis, loading a total of 2.6 ng of Gc or GcMAF per lane on 4-12% polyacrylamide gels. The gels were electrophoresed and transferred to NC membranes which were probed with a polyclonal rabbit anti-Gc antibody followed by goat anti-rabbit antibody conjugated to alkaline phosphatase and chemiluminescent detection.

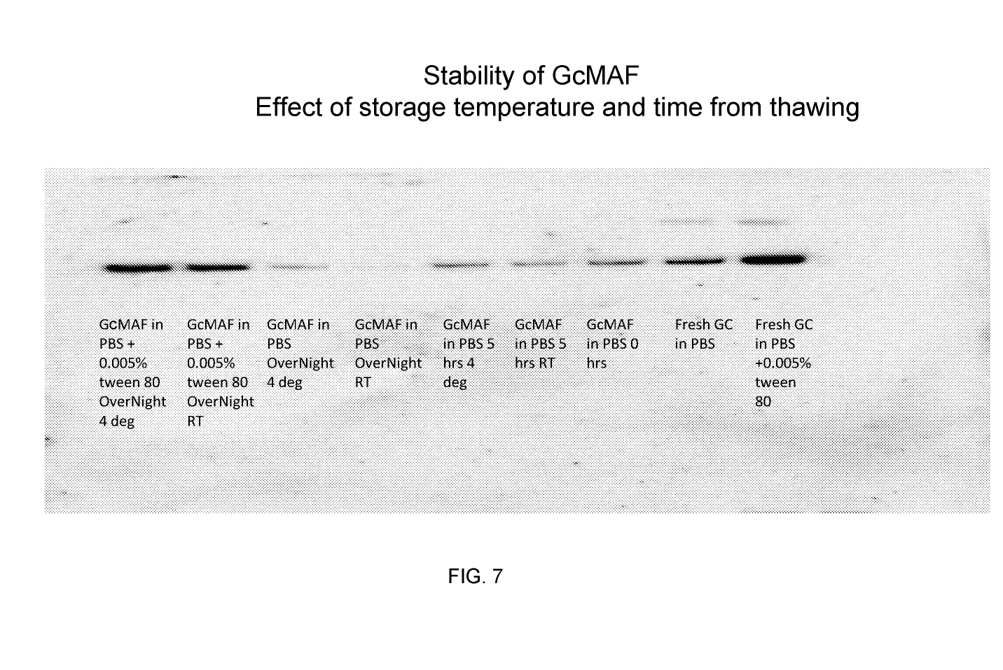

FIG. 7 shows the results of the western blot analysis. GcMAF samples containing TWEEN® 80 showed staining intensity similar to that of fresh GC samples containing TWEEN® 80 even after overnight storage at RT following thawing, while samples thawed and stored overnight at RT in PBS only were not detectable. A significant reduction in the intensity of the GcMAF band was noted even when GcMAF prepared in PBS was loaded on the polyacrylamide gel immediately after thawing (time 0; FIG. 7). Storing GcMAF at RT or at 4° C. in the absence of surfactants for 5 hrs resulted in additional reduction in GcMAF band intensity, indicating that GcMAF (in the absence of nonionic surfactants such as TWEEN® 80) is unstable even after short period of incubation. Thus, addition of TWEEN® 80 to GcMAF when undergoing freezing was shown to maintain GcMAF stability after thawing.

### Example 8

### Effect of Polysorbate 80 on Aggregate/Dimer Formation of GcMAF

GcMAF was prepared as described in Example 1 herein above. Briefly, Gc protein was purified from serum or plasma using 25-hydroxyvitamin D3-affinity chromatography. The purified Gc protein was incubated sequentially with immobilized β-galactosidase and sialidase to form GcMAF. The GcMAF was filtered through a 0.22 micron filter for sterilization. It was then diluted to a final concentration of 6 μg/ml in PBS pH 7.5 or in PBS containing 0.005% TWEEN® 80. The samples were subjected to three cycles of freezing and thawing to increase aggregate formation. The samples (in duplicates) were analyzed by western blot analysis, loading a total of 75 ng of GcMAF per lane on 4-12% polyacrylamide gels. The gels were electrophoresed and transferred to NC membranes which were probed with a polyclonal rabbit anti-Gc antibody followed by goat anti-rabbit antibody conjugated to alkaline phosphatase and chemiluminescent detection.

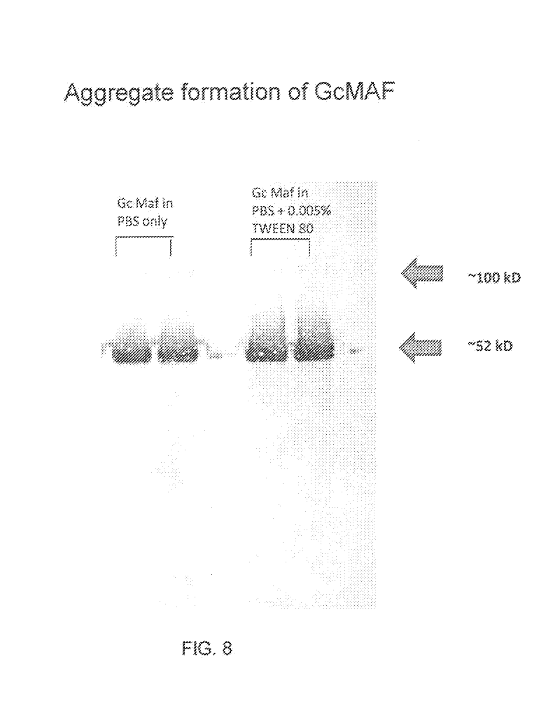

FIG. 8 shows that samples prepared in PBS containing 0.005% TWEEN® 80 showed a high intensity band of MW of approximately 52 kDa and a very faint band of MW of approximately 100 kDa (probably a dimer of the molecule). Samples prepared in the absence of TWEEN® 80 showed the same intensity of the 52 kDa band, however the high MW band of approximately 100 kDa was more intense. These results indicate that TWEEN® 80 is capable of reducing aggregate/dimer formation of GcMAF.

### Example 9

### Effect of Ionic Surfactants, Water-Soluble Polymers and Other Stabilizers on GcMAF Stability

GcMAF was prepared as described in Example 1 herein above. Briefly, Gc protein was purified from serum or plasma using 25-hydroxyvitamin D3-affinity chromatography. The purified Gc protein was incubated sequentially with immobilized β-galactosidase and sialidase to form GcMAF. The GcMAF was filtered through a 0.22 micron filter for sterilization. It was then diluted to a final concentration of 200 ng/ml in PBS pH 7.5 in the presence of the following surface active materials at the indicated concentrations: TWEEN® 80 (0.005%), SDS (0.02%), poly vinyl pyrrolydone (PVP) k90 (0.2%), polyvinyl alcohol (30-70 kD), sodium casein (0.2%), sodium alginate (0.2%), and hyaluronic acid (0.15%). The solutions were filtered and aseptically filled in 1 ml aliquots into 2 ml glass vials with rubber stoppers and aluminum caps and were kept at 37° C. at an upside down position. The samples (in duplicates) were analyzed at different time points by western blot analysis, loading a total of 2.6 ng of Gc or GcMAF per lane on 4-12% polyacrylamide gels. The gels were electrophoresed and transferred to NC membranes which were probed with a polyclonal rabbit anti-Gc antibody followed by goat anti-rabbit antibody conjugated to alkaline phosphatase and chemiluminescent detection.

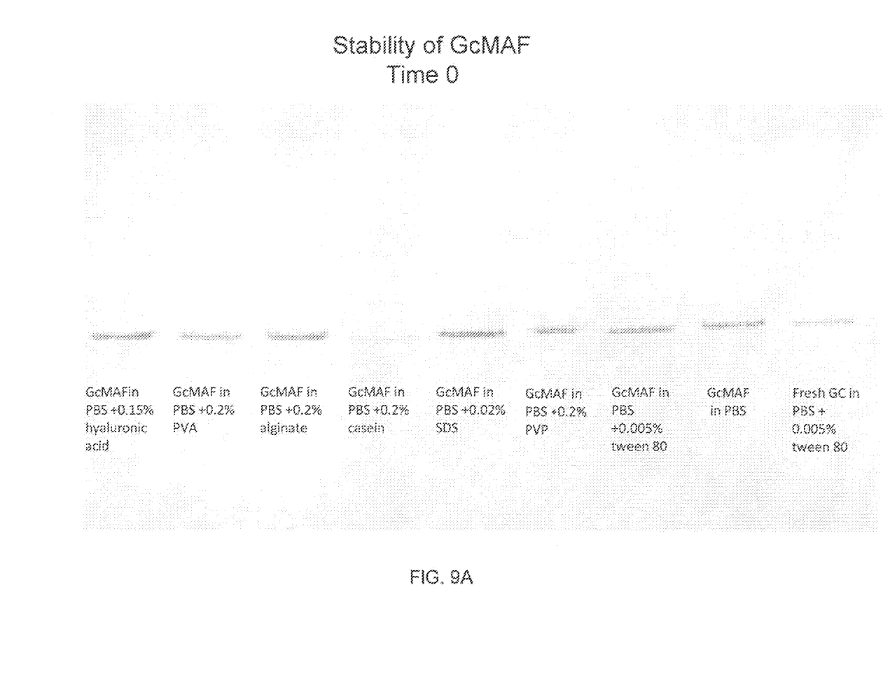

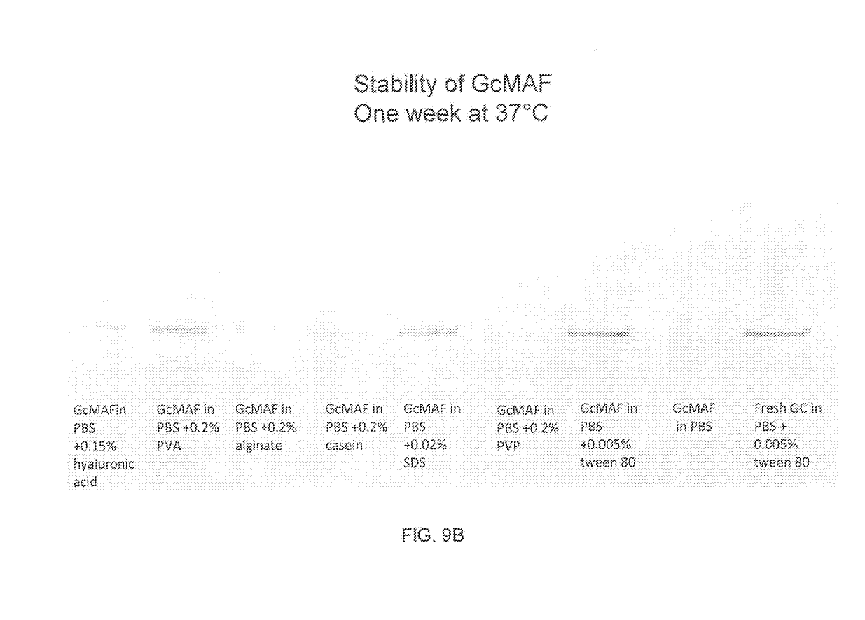

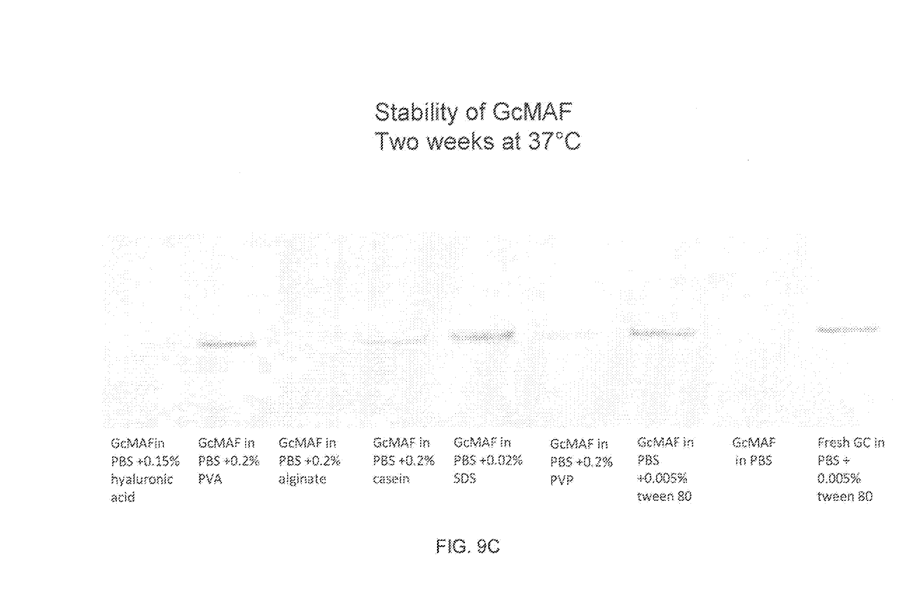

FIG. 9A shows western blot analysis performed at time point 0 (immediately after preparing the samples). All samples showed similar staining intensity as compared to the standard commercial Gc protein (Calbiochem), except of the casein containing sample which showed fainter signal.

FIG. 9B shows western blot analysis of GcMAF after incubation of 1 week at 37° C. GcMAF samples containing the non-ionic surfactant TWEEN® 80, the ionic surfactant SDS or the surface active polymer PVA showed clear visible bands with similar intensity to that obtained by the freshly diluted Gc protein. GcMAF samples prepared in PBS only or in PBS containing sodium alginate were not visible. Samples prepared in the presence of hyaluronic acid, PVP or casein were significantly fainter. Thus, PVP, a water-soluble polymer which does not have surface activity, was found to be less effective in stabilizing GcMAF.

FIG. 9C shows western blot analysis of GcMAF after incubation of 2 weeks at 37° C. GcMAF samples containing the non-ionic surfactant TWEEN® 80, the ionic surfactant SDS or the surface active polymer PVA showed clear visible bands with intensity similar to that obtained by the freshly diluted Gc protein. GcMAF samples prepared in PBS only or in PBS containing sodium alginate or hyaluronic acid were not visible. Samples prepared in PBS containing PVP or casein were significantly fainter than the freshly diluted Gc protein band.

These results indicate that the ionic surfactant SDS is highly effective in maintaining GcMAF stability. The water-soluble polymer PVA which has surface activity also shown to be effective in maintaining GcMAF stability. However, the polysaccharides hyaluronic acid and alginate were shown to be less effective in maintaining GcMAF in solution.

### Example 10

### Bioactivity of GcMAF in the Presence of TWEEN® 80

GcMAF was prepared as described in Example 1 herein above. Briefly, Gc protein was purified from serum or plasma using 25-hydroxyvitamin D3-affinity chromatography. The purified Gc protein was incubated sequentially with immobilized β-galactosidase and sialidase to form GcMAF. The GcMAF was filtered through a 0.22 micron filter for sterilization. It was then diluted to a final concentration of 200 ng/ml or 1 μg/ml in PBS pH 7.5 containing 0.005% TWEEN® 80. The solutions were filtered and aseptically filled in 1 ml aliquots into 2 ml glass vials with rubber stoppers and aluminum caps and were kept at −20° C. (straight position) for 0, 1, 3, 6, and 12 months or at 4° C. for 0, 1, 3, and 6 months at an upside down position. The samples were analyzed at the end of storage by a bioactivity test.

The bioactivity test was performed by measuring the release of hydrogen peroxide (H₂O₂) in RAW264.7 mouse macrophage cell line to determine macrophage activation following stimulation with GcMAF. Values of 2 folds and higher relative to controls without GcMAF were considered active. Release of hydrogen peroxide (>2 fold) was observed in all GcMAF samples tested at the above mentioned time points indicating that GcMAF maintained its biological activity for at least 6 months at 4° C. or 12 months when frozen at −20° C.

### Example 11

### Effect of Nonionic Surfactants or PVA on the Chemical and Biological Stability of GcMAF

GcMAF was prepared as described in Example 1 herein above. Briefly, Gc protein was purified from serum or plasma using 25-hydroxyvitamin D3-affinity chromatography. The purified Gc protein was incubated sequentially with immobilized β-galactosidase and sialidase to form GcMAF. The GcMAF was filtered through a 0.22 micron filter for sterilization. It was then diluted to a final concentration of 200 ng/ml in PBS pH 7.5, containing the following excipients at the indicated concentration: TWEEN® 80 (0.005%), TWEEN® 20 (0.005%), Poloxamer 188 (0.05%), N-dodecyl-beta-D-maltoside (0.01%), and the water-soluble polymer having surface activity—PVA (0.2%). The solutions were filtered and aseptically filled in 1 ml aliquots into 2 ml glass vials with rubber stoppers and aluminum caps and were kept at 37° C. at an upside down position. The samples (in duplicates) were analyzed by western blot analysis, loading a total of 2.6 ng of Gc or GcMAF per lane on 4-12% polyacrylamide gels. The gels were electrophoresed and transferred to NC membranes which were probed with a polyclonal rabbit anti-Gc antibody followed by goat anti-rabbit antibody conjugated to alkaline phosphatase and chemiluminescent detection.

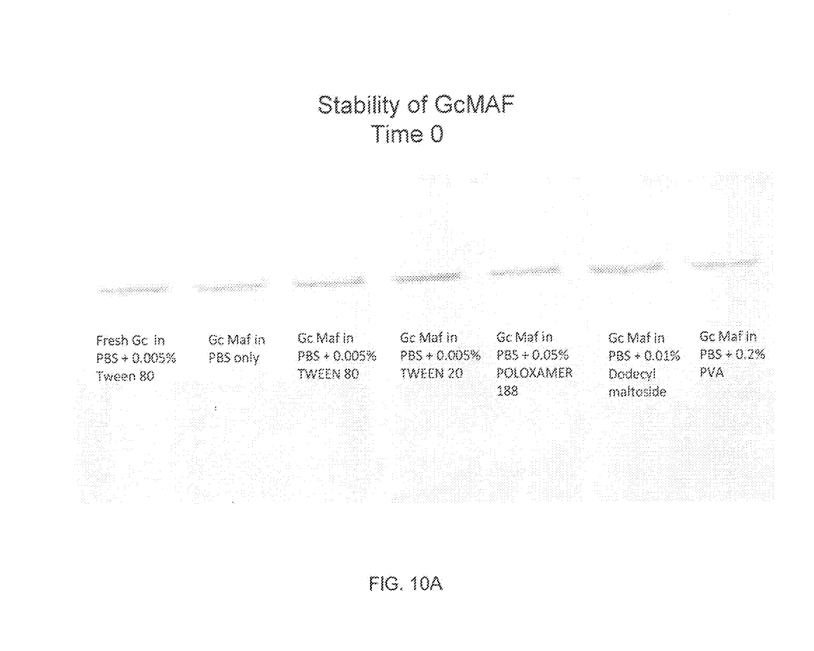

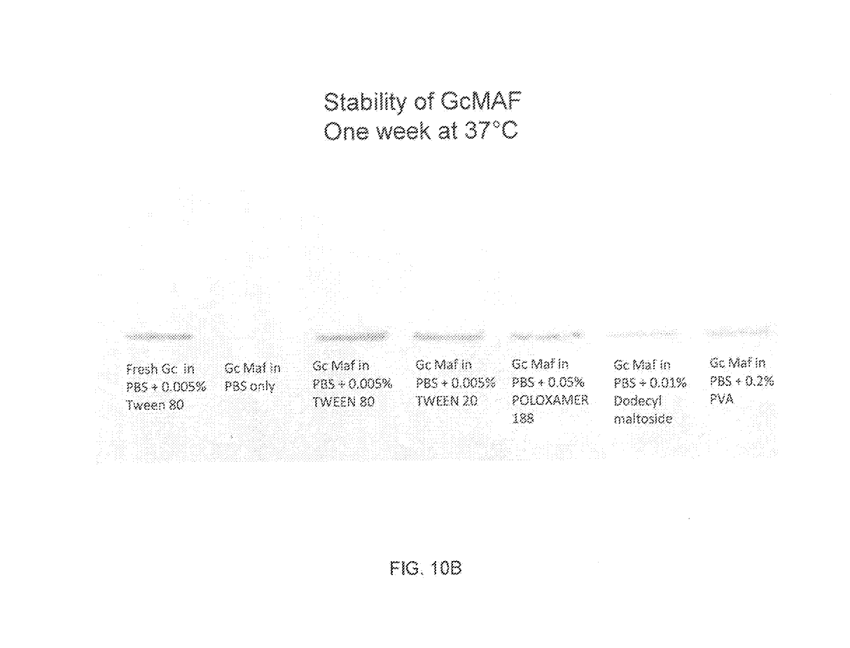

FIG. 10A shows western blot analysis performed at time point 0 (immediately after preparing the samples). All samples showed similar staining intensity as compared to the standard commercial Gc protein (Calbiochem).

FIG. 10B. shows western blot analysis of GcMAF or Gc samples incubated for one week at 37° C. GcMAF samples incubated in the presence of the indicated nonionic surfactants or the polymer PVA showed clear visible bands with similar intensity to that obtained by the freshly diluted Gc protein. GcMAF samples which were incubated in PBS only (in the absence of surfactants) showed significantly weaker staining compared to the freshly prepared standard GC or to the GcMAF samples incubated in the presence of the surfactants.

The bioactivity test was performed by measuring the release of hydrogen peroxide (H₂O₂) in RAW264.7 mouse macrophage cell line to determine macrophage activation following stimulation with GcMAF. Values of 2 folds and higher relative to controls without GcMAF were considered active. Release of hydrogen peroxide (>2 fold) was observed in the GcMAF samples containing the nonionic surfactants and the polymer after 1 week at 37° C. indicating that GcMAF maintained its biological activity.

It will be appreciated by persons skilled in the art that the present invention is not limited by what has been particularly shown and described herein above. Rather the scope of the invention is defined by the claims that follow.

1.-32. (canceled)

33. A pharmaceutical composition comprising stable Gc macrophage activating factor (GcMAF) or a biologically active variant or fragment thereof and at least one pharmaceutically acceptable excipient selected from the group consisting of a surfactant and a synthetic water-soluble polymer having surface activity.

34. The pharmaceutical composition according to claim 33, wherein the stable GcMAF has an N-acetylgalactosamine group linked to an amino acid residue and wherein said GcMAF is selected from the group consisting of a human GcMAF and an animal GcMAF.

35. The pharmaceutical composition according to claim 33, wherein the stable GcMAF comprises the amino acid sequence as set forth in any one of SEQ ID NOs: 1 to 3.

36. The pharmaceutical composition according to claim 33, wherein the stable fragment comprises the amino acid sequence corresponding to amino acids 400-435 of the Gc protein.

37. The pharmaceutical composition according to claim 33, wherein the stable fragment consists of the amino acid sequence as set forth in SEQ ID NO:4 or SEQ ID NO:5.

38. The pharmaceutical composition according to claim 33, wherein stable GcMAF is present at a concentration ranging from about 100 ng/ml to about 1 mg/ml.

39. The pharmaceutical composition according to claim 33, wherein the surfactant is selected from the group consisting of nonionic surfactants, anionic surfactants, cationic surfactants, amphoteric surfactants, and zwitterionic surfactants.

40. The pharmaceutical composition according to claim 39, wherein the nonionic surfactant is selected from the group consisting of sorbitan fatty acid esters, polyoxysorbitan fatty acid esters, polyoxyalkylene higher alcohol ethers, and polyoxyalkylene higher alcohol esters.

41. The pharmaceutical composition according to claim 39, wherein the nonionic surfactant is selected from the group consisting of polyoxyethylene sorbitol esters, polyoxyethylene isooctylphenyl ethers, polyoxyethylene nonylphenyl ethers, polyoxyethylene dodecyl ethers, octyl glucoside, and alkyl maltoside.

42. The pharmaceutical composition according to claim 39, wherein the nonionic surfactant is selected from the group consisting of polysorbate 80 (TWEEN® 80), polysorbate 60 (TWEEN® 60), polysorbate 20 (TWEEN® 20), N-dodecyl-beta-D-maltoside, Triton X-100, Brij 58, and Poloxamer 188.

43. The pharmaceutical composition according to claim 33, wherein the synthetic water-soluble polymer having surface activity is selected from the group consisting of polyvinyl alcohol, polypropylene oxide/ethylene oxide block co-polymers, copolymers of ethylene glycol/propylene glycol, poly-1,3-dioxolane, poly-1,3,6-trioxane, ethylene/maleic anhydride copolymer, propylene glycol homopolymers, and polyoxyethylated polyols.

44. The pharmaceutical composition according to claim 33, further comprising a tonicity agent, a buffering agent or a combination thereof.

45. The pharmaceutical composition according to claim 33, further comprising a pharmaceutically acceptable carrier or diluent.

46. The pharmaceutical composition according to claim 33, formulated in a form selected from the group consisting of a solution, a suspension, an emulsion, a powder, a tablet, and a capsule.

47. The pharmaceutical composition according to claim 33, comprising GcMAF, polysorbate 80, sodium chloride, phosphate buffer, and water, wherein the pH of the composition ranges between about 5 and about 8, and wherein the concentration of GcMAF ranges from about 100 ng/ml to 1 mg/ml.

48. A method of treating a disease or disorder associated with macrophage activation comprising administering to a subject in need of such treatment a therapeutically effective amount of the pharmaceutical composition according to claim 33.

49. The method according to claim 48, wherein the subject is a human being or an animal.

50. The method according to claim 48, wherein the pharmaceutical composition is suitable for administration via a parenteral route.

51. The method according to claim 48, wherein the disease or disorder associated with macrophage activation is selected from the group consisting of cancer, viral diseases, bacterial infections, autoimmune diseases, autism, and chronic fatigue syndrome.

52. The method according to claim 51, wherein the cancer is a solid tumor selected from the group consisting of sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, malignant synovioma, mesothelioma, Ewing's tumor leiomydsarcoma, rhabdomyosarcoma, colon carcinoma, pancreatic cancer, breast cancer, ovarian cancer, prostate cancer, squamous cell carcinoma, basal cell carcinoma, adenocarcinoma sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, oligodendroglioma, melanoma, neuroblastoma, and retinoblastoma.
